(19) United States
(12) Patent Application Publication
(10) Pub. No.: US 2026/0330732 A1
(43) Pub. Date: Oct. 22, 2026

(54) SYSTEMS AND METHODS FOR UNIFIED ENERGY CONVERSION IN COMPUTED TOMOGRAPHY IMAGING

(71) Applicant: GE Precision Healthcare LLC, Waukesha, WI (US)

(72) Inventors: Rajesh Langoju, Bengaluru (IN); Antony Jerald, Kottayam (IN); Bipul Das, Bengaluru (IN); Hemant Kumar Aggarwal, Bengaluru (IN); Phaneendra Yalavarthy, Bangalore (IN); Brian E. Nett, Wauwatosa, WI (US); Gopal B. Avinash, San Ramon, CA (US)

(21) Appl. No.: 19/185,106

(22) Filed: Apr. 21, 2025

(51) Int. Cl. G06T 12/30; A61B 6/00; A61B 6/03; A61B 6/46

(52) U.S. Cl. CPC G06T 12/30; A61B 6/032; A61B 6/461; A61B 6/5205; G06T 2211/408; G06T 2211/441

(57) ABSTRACT

A method includes receiving a computed tomography (CT) image acquired at a first energy level, obtaining input priors including an input energy spectrum corresponding to the first energy level, obtaining target priors including a target energy spectrum corresponding to a target energy level, processing the CT image through a neural network to generate at least one feature map, generating, using a feature attention generator, a feature conditioning vector based on the input priors and the target priors, adjusting features of the at least one feature map using the feature conditioning vector to generate adjusted features, and processing the adjusted features through the neural network to generate a transformed CT image at the target energy level. The feature attention generator enables precise control over feature adjustment based on global energy transformation requirements, allowing a single neural network to perform transformations across a continuous range of energy levels.

### TECHNICAL FIELD

Embodiments of the subject matter disclosed herein relate to medical imaging, and more particularly, to energy conversion in computed tomography (CT) imaging systems.

### BACKGROUND

In computed tomography (CT) imaging systems, an X-ray source emits an X-ray beam toward a subject or object, such as a patient. After attenuation by the subject, the X-ray beam impinges upon a detector array. An intensity of the attenuated beam radiation received at the detector array depends upon attenuation of the X-ray beam by the subject. Each detector element of the detector array produces a separate electrical signal which is transmitted to a data processing system for analysis and generation of a medical image.

CT scans at various energy levels may provide increased quality of tissue characterization and contrast quantification/visualization. For example, lower energy level images may increase contrast visualization of regions of interest (ROI) of a subject in the CT image. However, CT images at lower energy levels are also prone to noise and image artifacts which can decrease visualization of the ROI and reduce overall image quality.

Dual-energy CT imaging systems can generate CT images at different selected energy levels by obtaining projection data at multiple peak energy levels (e.g., 80 kVp and 140 kVp). For example, virtual monochromatic images may be generated at any desired energy level between 40 keV and 140 keV. However, dual-energy CT imaging systems require specialized hardware configurations, such as rapid kVp switching capabilities or dual source-detector arrangements, making them more complex and expensive than single-energy CT systems.

While single-energy CT imaging systems are more widespread, they traditionally cannot provide the multi-energy imaging capabilities of dual-energy systems. Deep learning approaches have been proposed to convert single-energy CT images to different energy levels. However, existing approaches require separate trained models for each specific energy level combination (e.g., separate models for converting 70 keV to 50 keV versus 70 keV to 40 keV). Additionally, these models are sensitive to contrast phase variations, requiring different models for different contrast phases. This results in a need for multiple individual models to handle various energy conversions and contrast phases, increasing computational overhead and system complexity.

Furthermore, the quality of energy conversion can depend significantly on the magnitude of the energy level change. Direct conversion between energy levels that are far apart (e.g., from 120 kVp to 30 keV) may result in unsatisfactory image quality. The complexity is further increased by the need to accurately transform both contrast-enhanced and non-contrast-enhanced tissues, while maintaining consistency across different display fields of view and reconstruction kernels.

### SUMMARY

The present disclosure provides systems and methods for unified energy conversion in CT imaging that at least partially address the above issues by providing a single “energy-to-energy” neural network model capable of performing multiple energy-level conversions while maintaining consistency across different contrast phases and display fields of view. In one embodiment, a method includes receiving a CT image acquired at a first energy level, obtaining input priors including an input energy spectrum corresponding to the first energy level, obtaining target priors including a target energy spectrum corresponding to a target energy level, processing the CT image through a neural network to generate at least one feature map, generating, using a feature attention generator, a feature conditioning vector based on the input priors and the target priors, adjusting features of the at least one feature map using the feature conditioning vector to generate adjusted features, and processing the adjusted features through the neural network to generate a transformed CT image at the target energy level.

This method addresses the limitations of existing approaches by providing a unified energy-to-energy neural network that can perform multiple energy-level conversions, rather than requiring separate models for each energy combination. The feature attention generator dynamically adjusts the network's behavior based on provided energy spectra and other prior information, enabling accurate energy conversion while maintaining image quality across different contrast phases and display fields of view.

In another embodiment, a method includes receiving a CT image acquired at a first energy level, obtaining input priors including an input energy spectrum corresponding to the first energy level, obtaining target priors including a target energy spectrum corresponding to a target energy level, processing the CT image through a neural network to generate a transformed CT image at the target energy level, wherein the neural network comprises an encoder pathway comprising a plurality of encoder levels, wherein each encoder level is configured to receive an input feature map and convolve the input feature map with one or more convolutional filters to generate an output encoder feature map, and a decoder pathway comprising a plurality of decoder levels corresponding to the plurality of encoder levels, wherein each decoder level is configured to receive an input decoder feature map comprising an up-convolved feature map from a lower decoder level concatenated with the output encoder feature map from a corresponding encoder level, generating at each decoder level, using a feature attention generator, a feature conditioning vector based on the input priors and the target priors, adjusting features of the input decoder feature map at each decoder level using a corresponding feature conditioning vector to generate adjusted features, convolving the adjusted features at each decoder level to generate an output decoder feature map, and outputting the transformed CT image based on an output decoder feature map from a highest level of the decoder pathway.

This method leverages a multi-level encoder-decoder architecture with feature attention at each decoder level to dynamically adjust the network's behavior based on both local and global image features. By incorporating skip connections and applying targeted feature scaling at multiple network depths, the system maintains fine anatomical details and structural consistency while performing energy conversion, particularly beneficial when converting between energy levels that are far apart where traditional approaches often suffer from significant quality degradation.

In another embodiment, a system includes a display device, one or more processors, and memory storing instructions executable by the one or more processors to receive a computed tomography CT image acquired at a first energy level, obtain input priors including an input energy spectrum corresponding to the first energy level, obtain target priors including a target energy spectrum corresponding to a target energy level, process the CT image through a neural network to generate a transformed CT image at the target energy level, wherein the neural network produces at least one feature map, generate, using a feature attention generator, a feature conditioning vector based on the input priors and the target priors, adjust features of the at least one feature map using the feature conditioning vector to generate adjusted features, generate the transformed CT image using the adjusted features, and display the transformed CT image on the display device.

This system enables integration of energy conversion capabilities into existing single-energy CT workflows. The feature attention mechanism, guided by physics-based energy spectra and other prior information, allows precise control over the transformation process at multiple scales, resulting in high-quality transformed images that maintain diagnostic value without requiring costly dual-energy CT hardware. This makes advanced energy-specific imaging capabilities accessible to facilities with conventional single-energy CT systems.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various aspects of this disclosure may be better understood upon reading the following detailed description and upon reference to the drawings in which:

FIG. 1 shows a pictorial view of a CT imaging system that incorporates disclosed embodiments;

FIG. 2 shows a block schematic diagram of the system illustrated in FIG. 1;

FIG. 3 shows an example image processing system, according to an embodiment of the present disclosure;

FIG. 4 illustrates an example neural network architecture for energy-to-energy conversion including feature attention generators, according to an embodiment of the present disclosure;

FIG. 5 schematically illustrates an example feature attention generator architecture, according to an embodiment of the present disclosure;

FIG. 6 is a flow chart illustrating a method for transforming CT images from a first energy level to a target energy level using feature conditioning vectors, according to an embodiment of the present disclosure;

FIG. 7 is a flow chart illustrating details of processing CT images through an encoder pathway, according to an embodiment of the present disclosure;

FIG. 8 is a flow chart illustrating details of processing feature maps through a decoder pathway informed by feature conditioning vectors, according to an embodiment of the present disclosure;

FIG. 9 is a flow chart illustrating a method for generating feature conditioning vectors, according to an embodiment of the present disclosure;

FIG. 10 is a flow chart illustrating a method for training a neural network to transform medical images between different energy levels, according to an embodiment of the present disclosure;

FIG. 11 is a flow chart illustrating a method for training a neural network to transform medical images between different energy levels using multiple target energy levels, according to an embodiment of the present disclosure;

FIG. 12 is a graphical representation of an X-ray spectrum showing the relationship between spectral amplitude and voltage at various operating voltages (kVp), according to an embodiment of the present disclosure;

FIG. 13 shows examples of encoded phase contrast timelines used as prior information for energy-to-energy conversion, according to an embodiment of the present disclosure;

FIG. 14 is a comparative display of CT images showing input, target, and predicted images at different energy levels, according to an embodiment of the present disclosure; and

FIG. 15 is a comparative visualization of the results of energy level conversion using the energy-to-energy neural network, according to an embodiment of the present disclosure.

### DETAILED DESCRIPTION

The following description relates to transforming images from one energy level to another energy level in CT imaging. In particular, the following description relates to a unified framework for converting CT images obtained at a first energy level to one or more different energy levels using a single neural network model that incorporates feature conditioning mechanisms guided by prior information relating to the input image and the one or more “target” images.

Unlike single-energy CT imaging systems, dual-energy CT imaging systems can generate images at any selected energy level between 40 keV and 140 keV by obtaining projection data at multiple peak energy levels (e.g., 80 kVp and 140 kVp). For example, virtual monochromatic images may be generated at any desired energy level by performing material decomposition using the dual-energy projection data. Thus, dual-energy CT imaging systems provide advantages for certain imaging tasks, such as improved contrast visualization and tissue characterization. However, dual-energy CT systems require specialized hardware configurations, making them more complex and expensive than single-energy CT systems.

While single-energy CT imaging systems are more widespread, they traditionally cannot provide the multi-energy imaging capabilities of dual-energy systems. Previous approaches have attempted to address this limitation by using deep learning to convert single-energy CT images to different energy levels. However, these approaches require separate trained models for each specific energy level combination (e.g., separate models for converting 70 keV to 50 keV versus 70 keV to 40 keV). Additionally, these models are sensitive to contrast phase variations, requiring different models for different contrast phases. This results in a need for multiple individual models to handle various energy conversions and contrast phases, increasing computational overhead and system complexity.

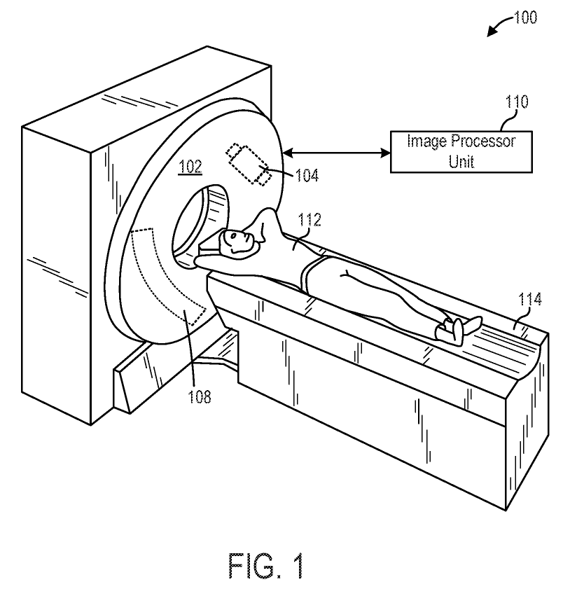

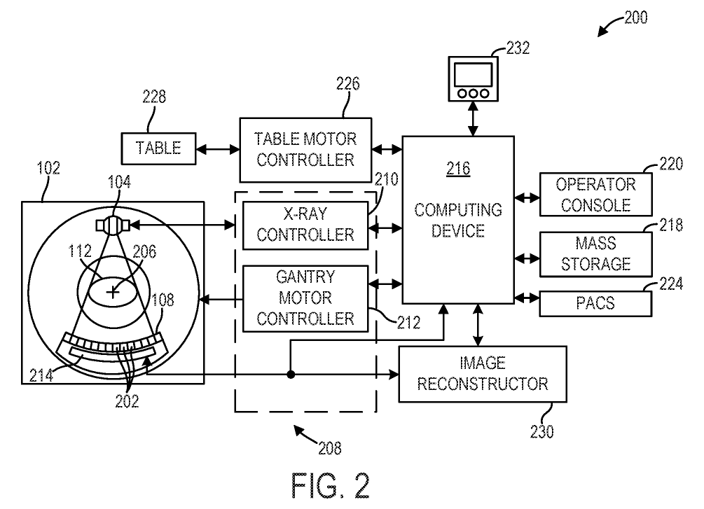

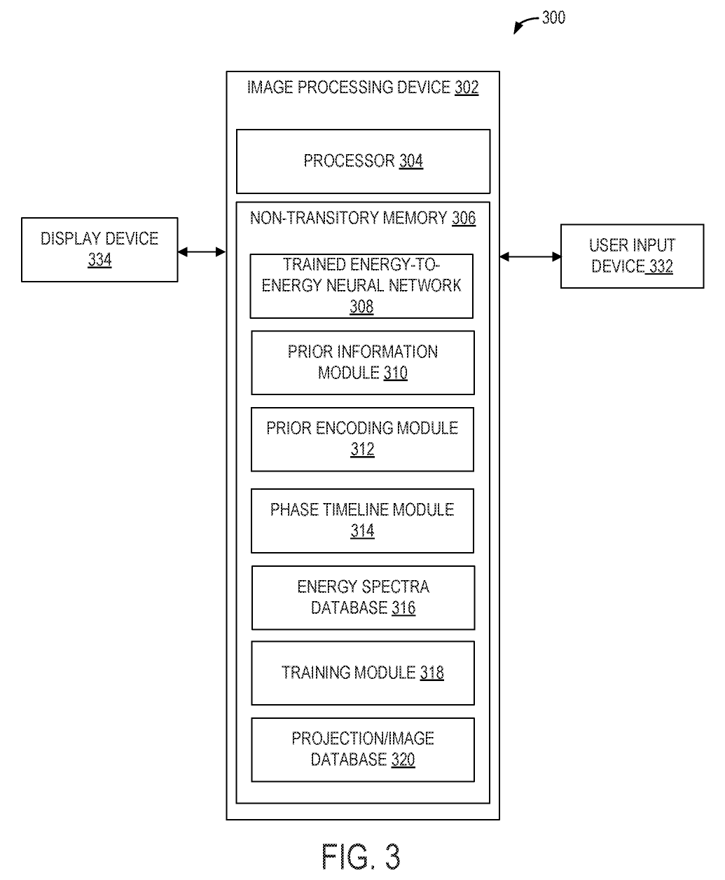

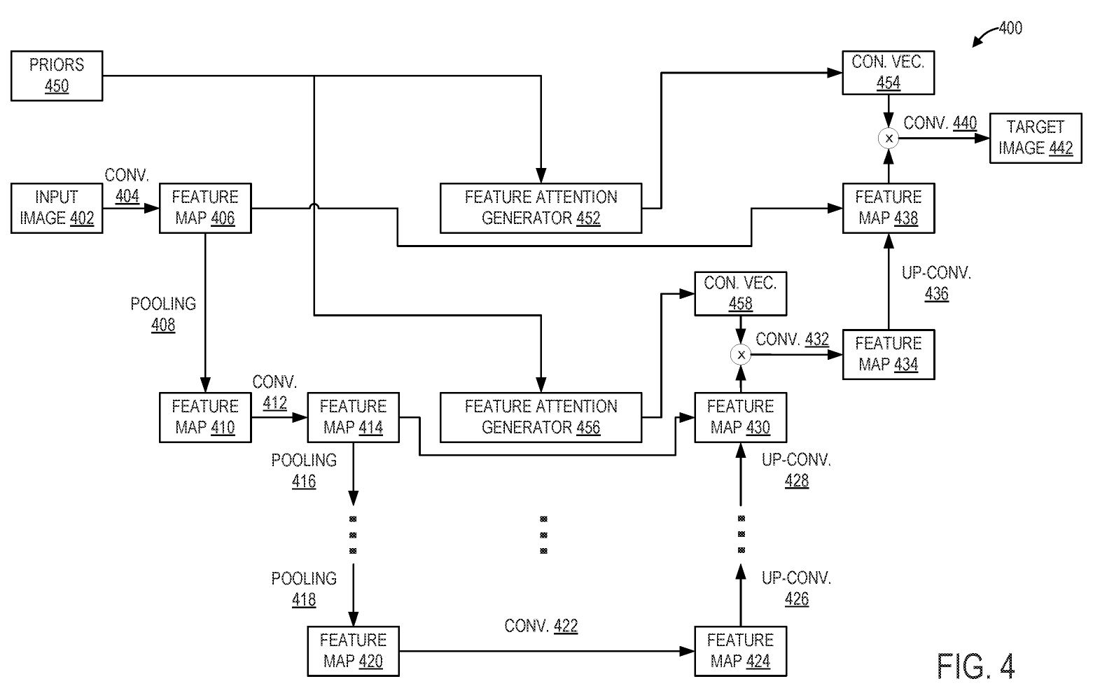

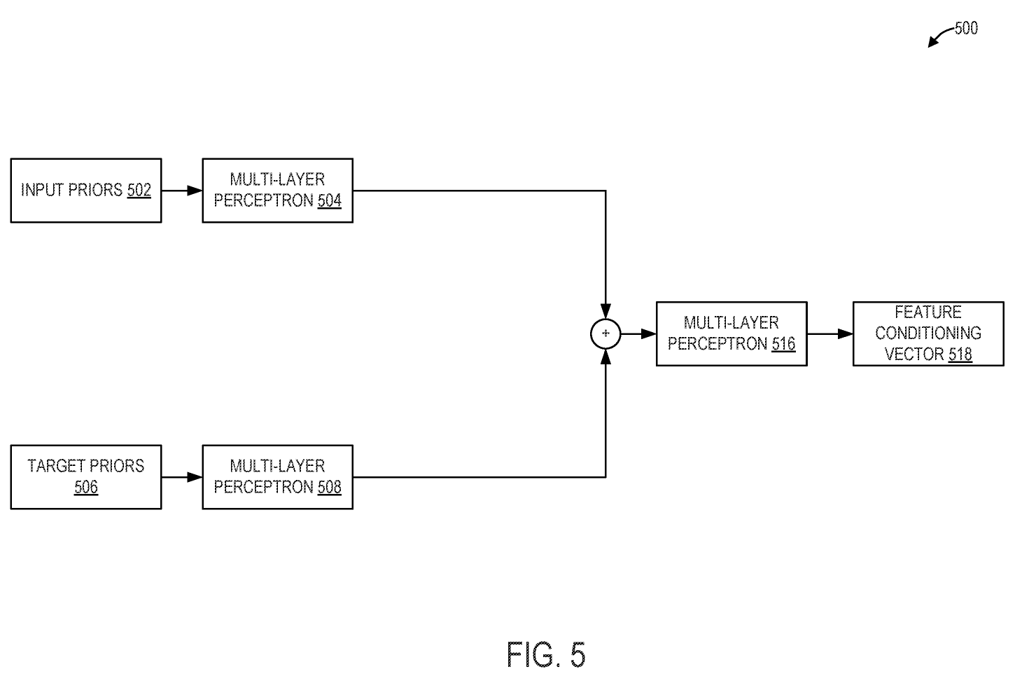

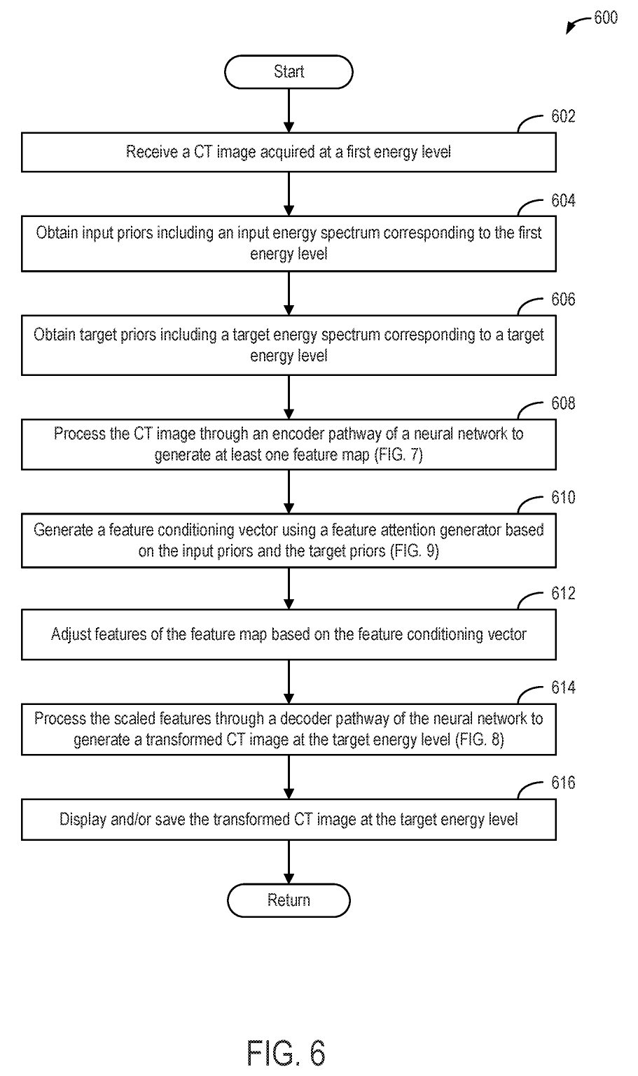

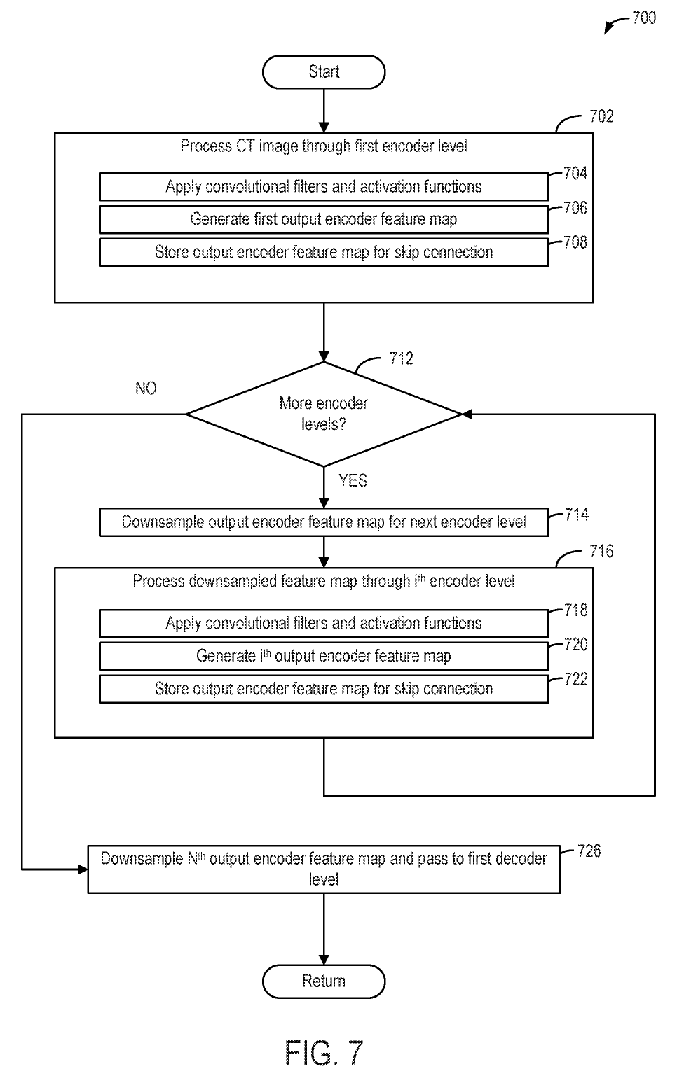

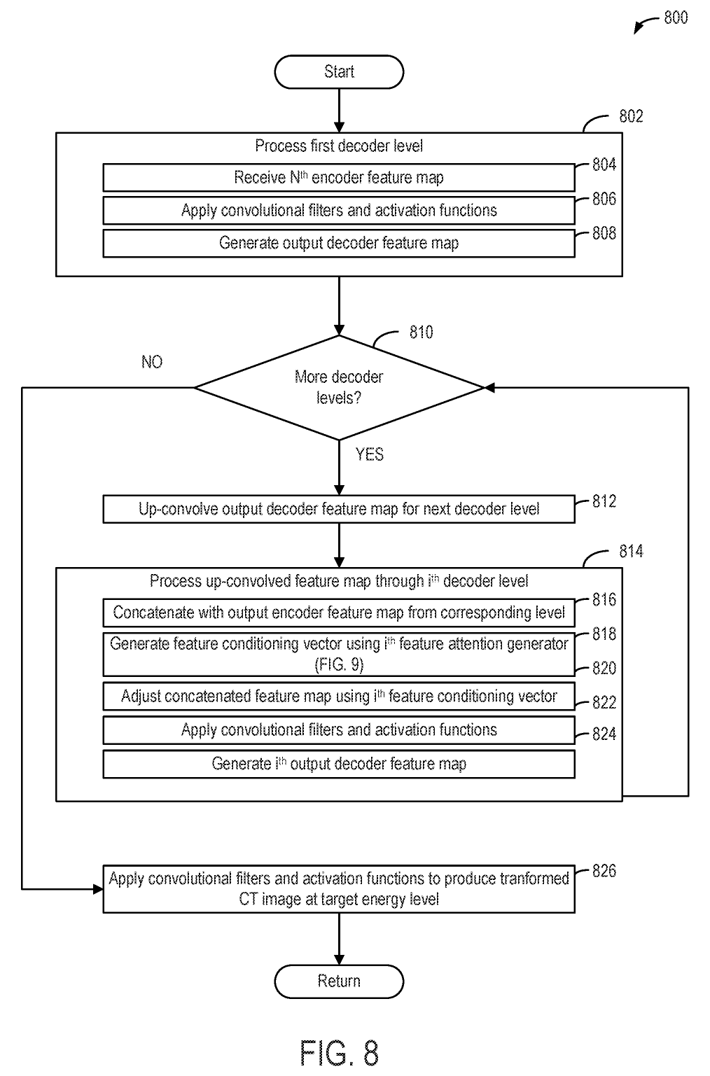

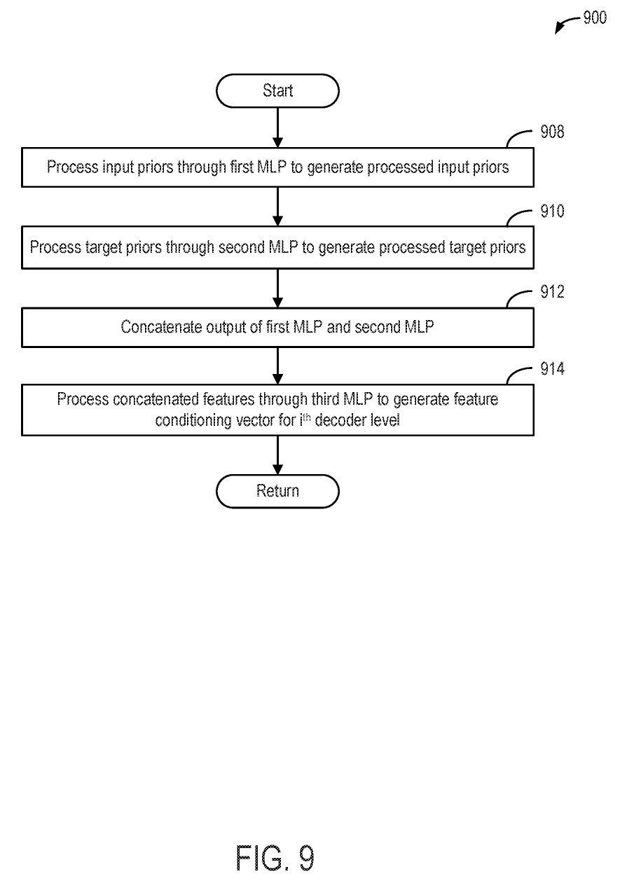

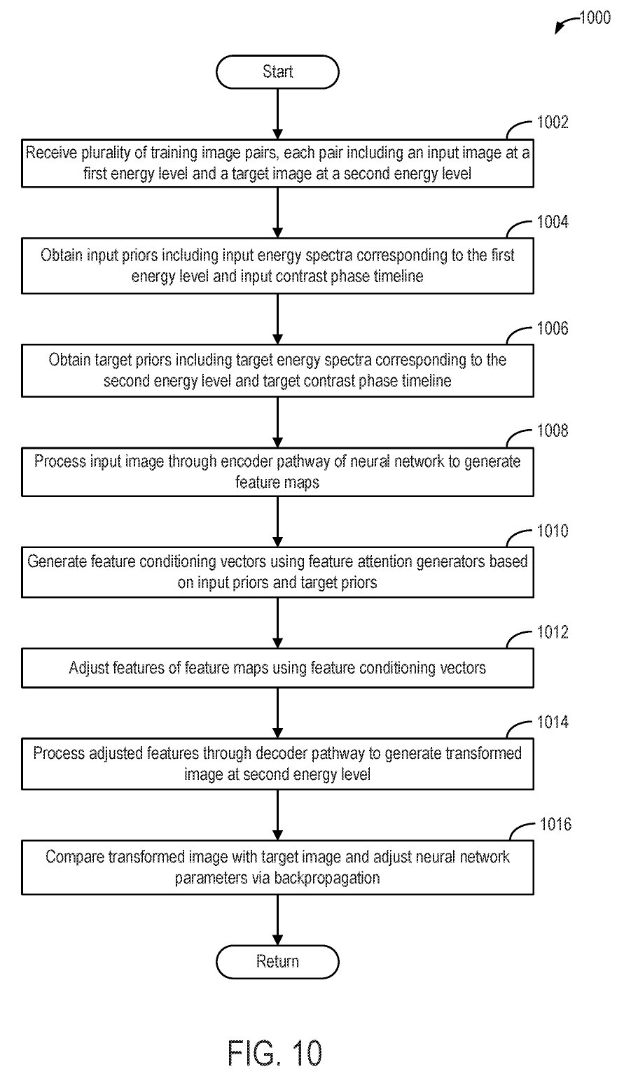

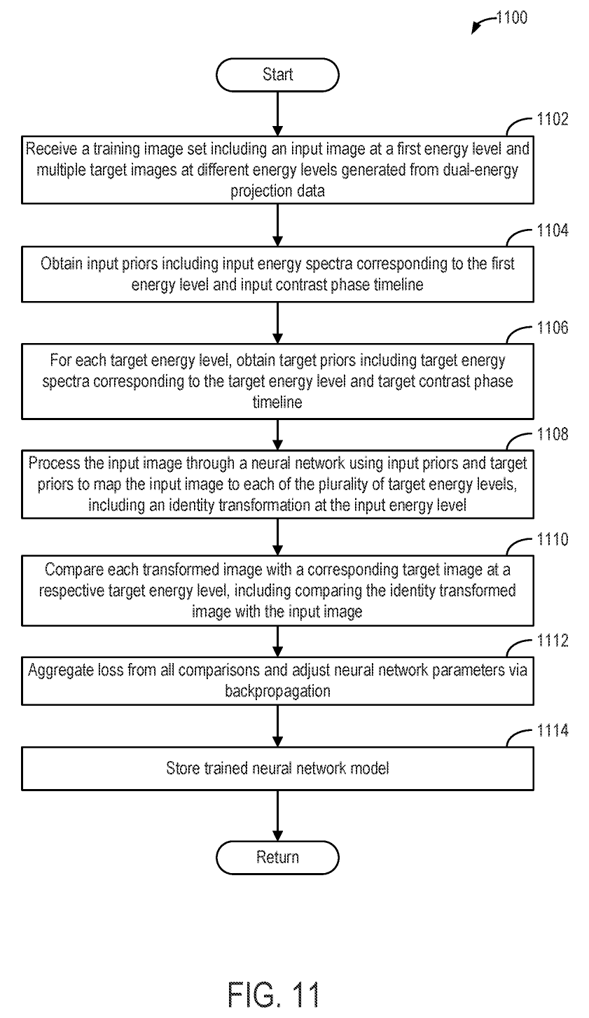

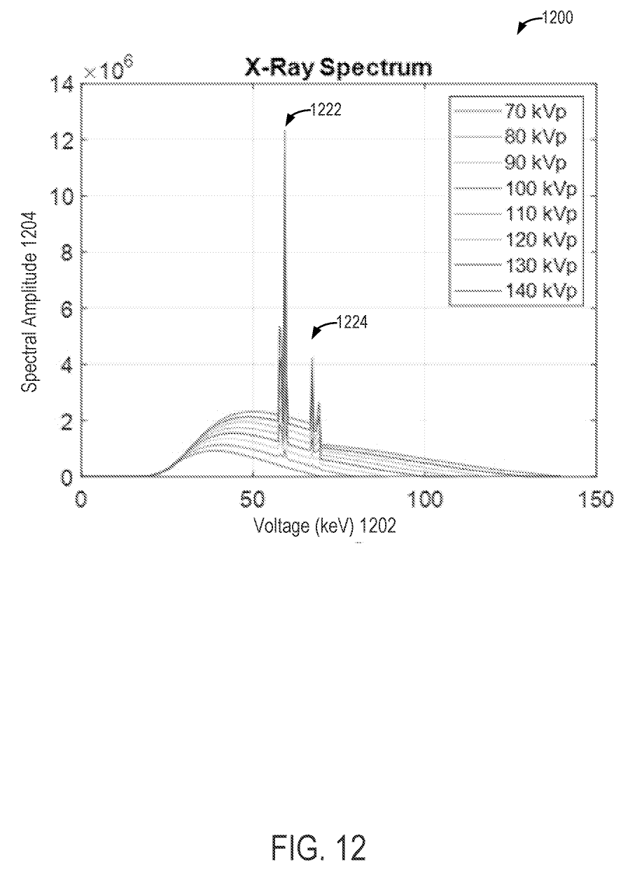

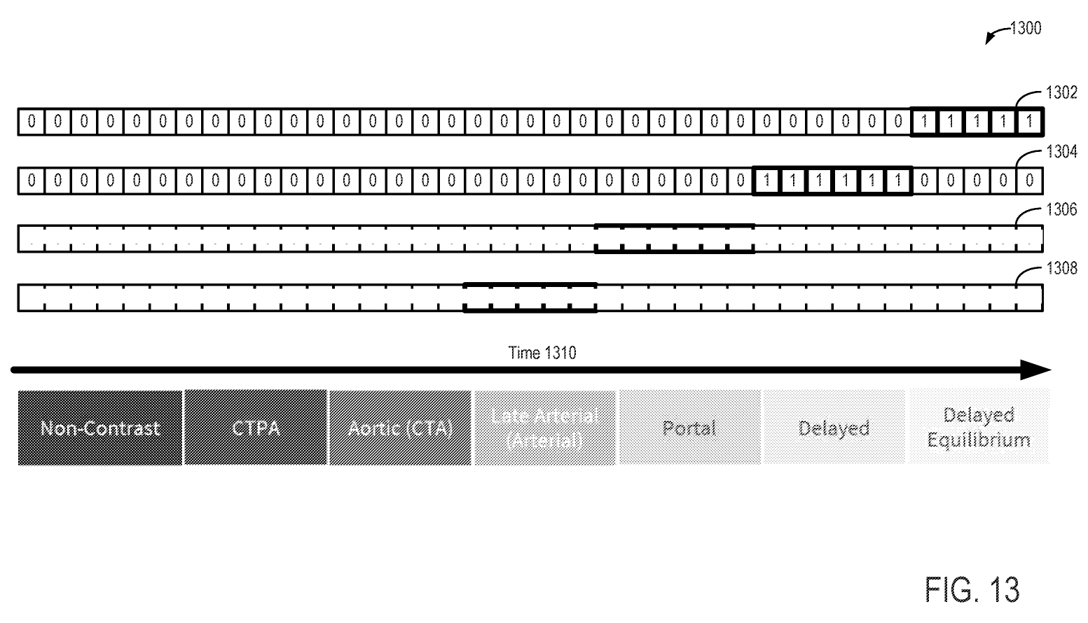

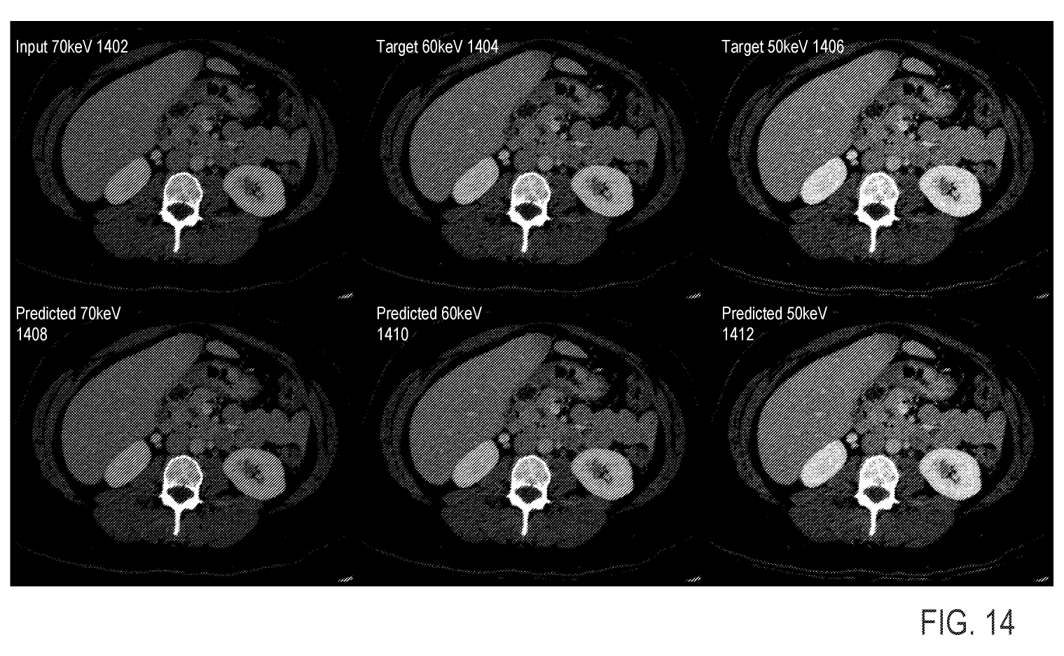

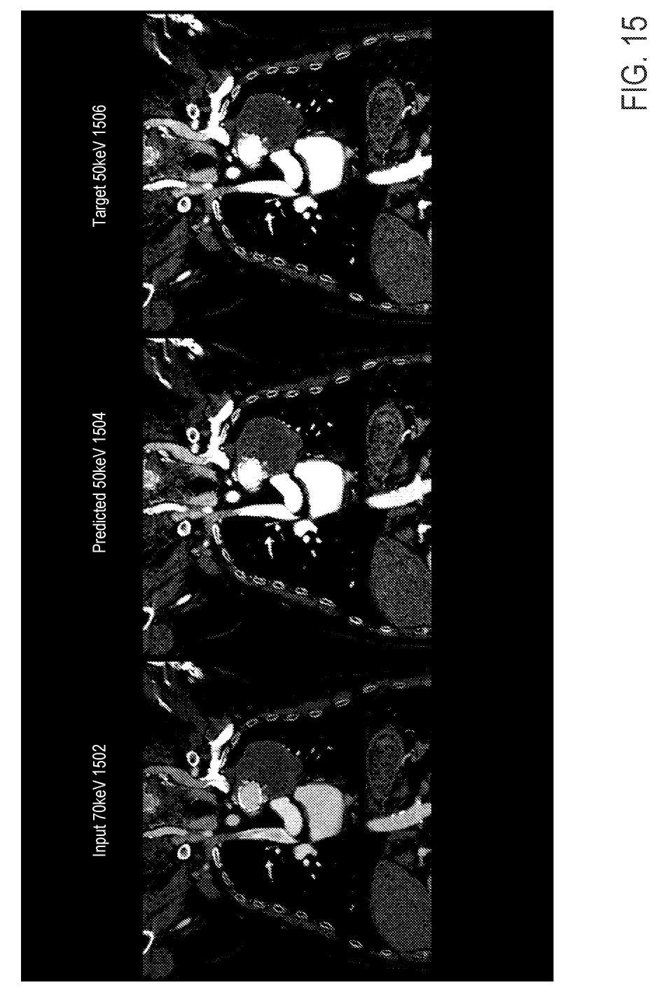

In one embodiment, a CT imaging system, as shown in FIGS. 1-2, may acquire CT images and process these images using an image processing system shown in FIG. 3. The image processing system includes a trained energy-to-energy neural network 308 that processes images through an encoder-decoder architecture illustrated in FIG. 4, incorporating feature attention generators detailed in FIG. 5. The feature attention generators dynamically adjust the network's behavior based on prior information including energy spectra stored in energy spectra database 316 and phase timeline information from phase timeline module 314. As illustrated in FIG. 6, the overall process includes receiving a CT image, obtaining input and target priors, processing through the encoder pathway, generating and applying feature conditioning, and processing through the decoder pathway to produce a transformed image. FIG. 7 details the encoder pathway processing steps, while FIG. 8 illustrates the decoder pathway operations including the application of feature conditioning vectors. FIG. 9 shows one embodiment of how feature conditioning vectors may be generated using the input and target priors. FIGS. 10-11 illustrate methods for training the neural network to transform medical images between different energy levels using feature conditioning vectors, with FIG. 10 showing the basic training approach and FIG. 11 demonstrating training with multiple target energy levels. FIG. 12 provides a graphical representation of X-ray spectra at various operating voltages, which serve as physics-based priors for the energy conversion process. FIG. 13 shows examples of encoded phase contrast timelines used as prior information, while FIGS. 14-15 present comparative visualizations of input, target, and predicted images at different energy levels, demonstrating the high-quality energy conversion achieved across different contrast phases and display fields of view.

This unified approach addresses the limitations of existing methods by providing a single neural network model capable of performing multiple energy-level conversions while maintaining consistency across different contrast phases and display fields of view. The feature conditioning mechanism, guided by physics-based energy spectra and other prior information, allows precise control over the transformation process at multiple scales, resulting in high-quality transformed images that maintain diagnostic value without requiring costly dual-energy CT hardware.

Referring to FIG. 1, a CT imaging system 100 for acquiring and processing medical imaging data at multiple energy levels is shown. The CT imaging system 100 is configured to acquire single-energy CT images and transform them to different energy levels using feature attention-based neural networks.

The CT imaging system 100 includes a gantry 102 that houses various imaging components. In one embodiment, the gantry 102 is configured to rotate around a central bore where a subject 112 is positioned during imaging. The gantry 102 supports and aligns the X-ray source 104 and detector array 108 in opposition to each other across the bore. In another embodiment, the gantry 102 includes mounting hardware and electrical connections for multiple X-ray sources and detector arrays to enable dual-energy imaging capabilities.

The X-ray source 104 is configured to emit an X-ray beam toward the subject 112 for imaging. In one embodiment, the X-ray source 104 operates at a single peak kilovoltage (kVp) level, such as 120 kVp, which produces X-ray spectra approximately equivalent to 70keV monochromatic X-rays. In another embodiment, the X-ray source 104 may enable rapid kVp switching between multiple energy levels. The X-ray source 104 is mechanically and electrically coupled to the gantry 102 and receives control signals from the image processor unit 110 to coordinate X-ray generation with detector array 108 readout.

The detector array 108 is positioned opposite the X-ray source 104 across the bore of the gantry 102 to detect X-rays that have passed through the subject 112. In one embodiment, the detector array 108 includes multiple rows of detector elements that produce separate electrical signals proportional to the attenuated X-ray intensity at each element. In another embodiment, the detector array 108 may comprise energy-integrating detectors that measure total deposited energy. The detector array 108 is electrically coupled to the image processor unit 110 to transmit the detector signals for image reconstruction and processing.

The image processor unit 110 receives and processes the detector signals to reconstruct CT images. In one embodiment, the image processor unit 110 includes the trained energy-to-energy neural network that processes images through an encoder-decoder architecture with feature attention generators. The neural network receives input priors including energy spectra and phase timeline information to guide the energy level conversion process. In another embodiment, the image processor unit 110 includes the prior information module, prior encoding module, and phase timeline module for preprocessing image data and associated prior information before neural network processing.

The subject 112 may be a patient, phantom, or other object positioned on the table 114 for CT imaging. In one embodiment, the subject 112 is a patient receiving contrast agent for enhanced visualization of specific anatomical structures, where the contrast agent produces different enhancement patterns across multiple contrast phases. In another embodiment, the subject 112 is a calibration phantom with known material compositions used for validating the energy conversion capabilities of the system.

The table 114 supports and positions the subject 112 within the bore of the gantry 102. In one embodiment, the table 114 is motorized to move the subject 112 along the z-axis (into/out of the bore) to enable helical scanning, where projection data is acquired as the table 114 moves continuously through the gantry 102. In another embodiment, the table 114 includes positioning controls to adjust height, tilt, and lateral position to optimize subject positioning relative to the X-ray source 104 and detector array 108. The table 114 movements are coordinated with the image acquisition process through control signals from the image processor unit 110.

FIG. 2 illustrates an exemplary imaging system 200 similar to the CT imaging system 100 of FIG. 1. The imaging system 200 is configured for imaging the subject 112. In one embodiment, the imaging system 200 includes the detector array 108 (see FIG. 1). The detector array 108 further includes a plurality of detector elements 202 that together sense the X-ray beams that pass through the subject 112 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 112 for acquiring desired projection data. Accordingly, the gantry 102 and the components mounted thereon may be configured to rotate about a center of rotation 206 for acquiring the projection data at a single energy level.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 112. The processed data are commonly called projections.

The detector elements 202 of the detector array 108 may include energy-integrating detectors that measure total deposited energy from the X-ray beams. The acquired projection data is used to reconstruct CT images at the acquisition energy level.

Once reconstructed, the CT image reveals internal features of the subject 112. The image may be transformed to appear as if acquired at different energy levels using the energy-to-energy neural network described herein. The transformed images may provide enhanced contrast visualization while maintaining low noise and artifacts compared to images directly acquired at lower energy levels.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The data sampled and digitized by the DAS 214 is transmitted to a computer or computing device 216. In one example, the computing device 216 stores the data in a mass storage device or storage device 218. The storage device 218 may also store energy spectra information, phase timeline data, and other prior information (DFOV) used by the energy-to-energy neural network.

The computing device 216 includes the trained energy-to-energy neural network that processes images through an encoder-decoder architecture with feature attention generators. The neural network receives input priors including energy spectra and phase timeline information to guide the energy level conversion process. The computing device 216 may also include the prior information module, prior encoding module, and phase timeline module for preprocessing image data and associated prior information before neural network processing.

The operator console 220 allows an operator to control scanning parameters and view both original and transformed images. The operator may select desired target energy levels for image transformation through a user interface of the operator console 220.

The imaging system 200 may be coupled to multiple displays, printers, workstations, and/or similar devices for viewing both original and transformed images. The transformed images may be transmitted across networks to allow remote viewing and analysis.

In one embodiment, the imaging system 200 either includes, or is coupled to, a picture archiving and communications system (PACS) 224. The PACS 224 may store both original images and transformed images generated at different target energy levels.

The computing device 216 uses the operator-supplied and/or system-defined commands and parameters to operate a table motor controller 226, which controls a motorized table 228. The table motor controller 226 moves the table 228 to position the subject 112 in the gantry 102 for acquiring projection data.

The image reconstructor 230 uses the sampled and digitized Xray data to perform high-speed reconstruction of the CT image at the acquisition energy level. The reconstructed image may then be processed by the energy-to-energy neural network to generate transformed images at different target energy levels.

The computing device 216 may transmit both the original reconstructed images and the transformed images to a display device 232 communicatively coupled to the computing device 216. The display device 232 may show the original and transformed images side-by-side to allow comparison of tissue contrast and detail at different energy levels.

Referring to FIG. 3, an image processing system 300 for processing energy-based imaging data is shown. The image processing system 300 includes an image processing device 302 configured to receive and process CT imaging data at multiple energy levels. In one embodiment, image processing device 302 is incorporated into a CT imaging system as an image processor unit or computing device. In another embodiment, at least a portion of image processing device 302 is disposed at a separate device communicably coupled to the CT imaging system via wired or wireless connections.

Image processing device 302 includes a processor 304 configured to execute machine readable instructions stored in non-transitory memory 306. In one embodiment, processor 304 may be single core or multi-core, and programs executed thereon may be configured for parallel or distributed processing. In another embodiment, processor 304 may include components distributed throughout multiple devices configured for coordinated processing. The processor 304 may also be virtualized and executed by remotely-accessible networked computing devices in a cloud computing configuration.

Non-transitory memory 306 stores various modules and databases for implementing energy conversion in CT imaging. The memory 306 includes a trained energy-to-energy neural network 308 that processes images through various neural network architectures incorporating feature attention generators. In one embodiment, the neural network 308 receives input priors and target priors to guide the energy level conversion process through feature conditioning vectors. In another embodiment, the neural network 308 may implement an encoder-decoder architecture with skip connections between corresponding encoder and decoder levels to maintain fine anatomical details during energy conversion, where feature conditioning vectors can adjust feature maps in both encoder and decoder pathways.

The prior information module 310 manages various types of prior information used to guide the energy conversion process. In one embodiment, the prior information module 310 handles input priors including energy spectra, contrast phase timelines, display field of view values, and reconstruction kernel types. In another embodiment, the module 310 processes target priors including target energy spectra and matching or different contrast phase information.

The prior encoding module 312 converts prior information into features that influence the training and inference processes. In one embodiment, the module 312 encodes energy spectra, phase timelines, and other priors into feature vectors used by the feature attention generators. In another embodiment, the prior encoding module 312 processes priors through multiple MLPs to generate encoded features for feature conditioning vector generation.

The phase timeline module 314 manages contrast phase timeline information for both training and inference. In one embodiment, the module 314 maintains phase timeline data indicating contrast enhancement patterns across different anatomical structures over time. In another embodiment, the module 314 provides phase timeline priors to enable the network to maintain consistency across different contrast phases.

The energy spectra database 316 stores energy spectrum information for different kVp and keV levels. In one embodiment, the energy spectrum database 316 contains X-ray spectra corresponding to common acquisition energy levels like 120 kVp and virtual monochromatic energy levels from 40-140 keV. In another embodiment, the database 316 provides spectrum priors to guide energy-specific transformations in the neural network.

The training module 318 implements procedures for training the energy-to-energy neural network. In one embodiment, the module 318 trains using pairs of images at different energy levels generated from dual-energy projection data. In another embodiment, the module 318 updates network parameters based on comparing transformed images to ground truth images while incorporating prior information.

The projection/image database 320 stores CT projection data and reconstructed images. In one embodiment, the projection/image database 320 contains training datasets including paired images at different energy levels and contrast phases. In another embodiment, the database 320 stores transformed images generated by the neural network for validation and clinical use.

The display device 334 enables visualization of original and transformed CT images. In one embodiment, display device 334 comprises a computer monitor showing side-by-side comparisons of images at different energy levels. In another embodiment, the display device 334 presents transformed images with enhanced contrast for improved diagnostic visualization.

The user input device 332 enables user interaction with the image processing system. In one embodiment, user input device 332 includes a touchscreen, keyboard, and mouse for selecting images and specifying target energy levels for transformation. In another embodiment, the input device 332 allows users to adjust processing parameters and view results interactively.

Referring to FIG. 4, an image processing architecture 400 is shown that incorporates feature attention mechanisms and multi-scale processing for transforming a CT image from a first energy level to a target energy level. The architecture implements an encoder-decoder framework with multiple encoding and decoding levels, where feature attention generators are incorporated at each decoder level to dynamically adjust features based on input and target energy spectra and other prior information. The architecture enables accurate energy conversion while maintaining anatomical details and image quality across different contrast phases and display fields of view through the coordinated use of multi-scale processing and feature attention mechanisms. This unified approach eliminates the need for separate models for each energy combination and contrast phase, reducing computational overhead and system complexity while maintaining consistent image quality. The feature attention generators, guided by physics-based energy spectra and other prior information, allow precise control over the transformation process at multiple scales, resulting in high-quality transformed images that maintain diagnostic value without requiring costly dual-energy CT hardware.

The encoder pathway begins with a first encoding level that receives an input CT image 402 acquired at a first energy level (e.g., 70 keV or 120 kVp). The input image 402 may be a 2D slice image, a 3D volume rendering, or a maximum intensity projection (MIP) image obtained from projection data. The input image 402 is processed through convolution layer 404, which applies multiple learned convolutional filters and non-linear activation functions to generate an initial feature map 406 capturing low-level image features such as edges, textures, and intensity gradients characteristic of different tissue types and contrast-enhanced regions. For example, the convolution layer 404 may apply 3x3 or 5x5 convolutional kernels followed by ReLU activation functions to extract meaningful features while maintaining spatial relationships. A pooling layer 408 performs spatial downsampling on feature map 406, in some embodiments using max pooling operations with 2x2 windows and stride 2 to reduce spatial dimensions while preserving dominant features such as high-contrast boundaries and enhanced vessel structures. The pooling operations help achieve translation invariance and reduce computational complexity in deeper network layers. A feature attention generator 452 processes prior information 450 including input energy spectra characterizing X-ray attenuation properties at the first energy level, target energy spectra defining desired attenuation characteristics, contrast phase timelines indicating temporal contrast agent dynamics, and display field of view parameters specifying image reconstruction settings to generate feature conditioning vector 454. The feature conditioning vector 454 will be used to adjust features at the corresponding highest decoder level, enabling the network to selectively modify different feature channels based on the specific energy conversion requirements and imaging conditions. The prior information 450 may also include reconstruction kernel types, anatomical region indicators, and other acquisition parameters that influence the energy transformation process.

A second encoding level receives the downsampled output from pooling layer 408 as feature map 410, which maintains structural and contrast information while operating at a reduced spatial resolution. The feature map 410 is processed through convolutional layer 412, which applies additional sets of learned filters, which in some embodiments may include multiple 3×3 and 5×5 convolutional kernels followed by non-linear activation functions such as ReLU or LeakyReLU, to generate feature map 414 capturing intermediate-level image features and energy-specific characteristics. These intermediate features may include tissue boundaries, contrast-enhanced vessel structures, organ textures, and localized energy-dependent attenuation patterns characteristic of different anatomical regions. Pooling layer 416 performs further spatial downsampling, typically using max pooling with 2×2 windows and stride 2, to reduce computational complexity while maintaining a rich feature set that preserves dominant activation patterns. A feature attention generator 456 processes the prior information 450 to generate feature conditioning vector 458 for dynamically adjusting features at the corresponding decoder level based on the desired energy transformation parameters. The feature conditioning vector 458 may be computed through processing of energy spectra and phase information through dedicated MLPs, and generation of channel-wise conditioning factors that selectively modify different feature channels based on the specific energy conversion requirements.

The encoder pathway continues through additional encoding levels, as indicated by the ellipsis between pooling layer 416 and pooling layer 418, with each successive level following a similar pattern of feature map generation, convolution, and pooling operations to progressively capture higher-level image features at different scales. These additional levels, which may in some embodiments may number between 3 to 5 depending on the input image resolution and desired feature hierarchy, enable the network to learn increasingly abstract representations combining both local and global image characteristics. Each level reduces spatial dimensions while increasing the number of feature channels, in some embodiments doubling from 64 to 128 to 256 and so on, allowing the network to capture more complex patterns and relationships. The bottom encoding level generates feature map 420, which is processed through convolution layer 422 using specialized kernels designed for capturing global contextual information to produce feature map 424. This final feature map 424 may in general contain more abstract representations of the input image and energy-specific characteristics than preceding encoding levels, including anatomical context, global contrast distribution patterns, and high-level energy-dependent imaging properties that guide the subsequent decoding process. In some embodiments, the lowest level of the energy-to-energy neural network architecture 400 operates at 1/16 or 1/32 of the original image resolution, with feature channels numbering 512 or 1024 to maintain rich representational capacity despite the reduced spatial dimensions.

The decoder pathway begins at the lowest level, where feature map 424 undergoes up-convolution through up-convolution layer 426 to increase spatial dimensions. In some embodiments, the up-convolution operations use transposed convolution with learnable filters of size 2×2 or 4×4 and stride 2, effectively doubling the spatial dimensions while reducing the number of feature channels. Additional decoder levels follow, as indicated by the ellipsis, with each level corresponding to an encoding level and incorporating skip connections to preserve fine spatial details by incorporating the output encoder feature map from the corresponding encoder level. In some embodiments, the number of decoder levels matches the number of encoder levels, and may range from 4 to 6 levels depending on the input image resolution. The penultimate decoder level receives an up-convolved feature map via up-convolution layer 428, which may employ similar transposed convolution operations but with different numbers of output channels, typically half the number of the previous level. The up-convolved feature map is concatenated with feature map 414 from the corresponding encoder level through skip connections to produce feature map 430, enabling direct access to earlier spatial information that may have been lost during downsampling. The concatenated features of feature map 430 are adjusted using feature conditioning vector 458 generated by feature attention generator 456 to selectively modify features relevant to the target energy level. In some embodiments, the feature conditioning vector 458 contains channel-wise conditioning factors computed based on global energy transformation requirements. The adjusted features are then processed through convolution layer 432, which may employ multiple 3×3 convolutional filters followed by batch normalization and ReLU activation, to produce feature map 434 with refined local details while maintaining energy-specific characteristics.

Feature map 434 is up-convolved through up-convolution layer 436 to the highest decoder level to restore full spatial resolution. In some embodiments, this final up-convolution employs a number of output channels matched to maintain consistency with the corresponding encoder level features. At this level, the up-convolved features are concatenated with feature map 406 from the first encoding level through skip connections, to produce feature map 438. The feature channels of feature map 438 are then adjusted using feature conditioning vector 454 from feature attention generator 452, which modifies feature weights based on the input and target energy characteristics. In some embodiments, the feature conditioning vector 454 is computed using multiple MLPs processing energy spectra information. The adjusted features of feature map 438 are then processed through convolution layer 440, which may employ a final set of convolutional filters (e.g., 32 or 64 3×3 filters) followed by a 1×1 convolution to generate the final target image 442 at the desired target energy level (e.g., 40 keV, 50 keV, or 60 keV). In some embodiments, additional post-processing operations such as residual connections or adaptive normalization may be applied to enhance the final output quality.

The energy-to-energy neural network architecture 400 detailed in FIG. 4 provides several technical advantages over existing approaches. The multi-level encoder-decoder framework with feature attention at each decoder level enables dynamic adjustment of the network's behavior based on both local and global image features. By incorporating skip connections between corresponding encoder and decoder levels and applying targeted feature conditioning through feature conditioning vectors at multiple network depths, the system maintains fine anatomical details and structural consistency while performing energy conversion. This is particularly beneficial when converting between energy levels that are far apart, where traditional approaches often suffer from significant quality degradation. The feature attention generators, guided by physics-based energy spectra and other prior information, allow precise control over the transformation process at multiple scales. Additionally, the unified approach eliminates the need for separate models for each energy combination and contrast phase, reducing computational overhead and system complexity while maintaining consistent image quality across different display fields of view. The architecture's ability to process both input and target energy spectra as priors enables accurate energy-specific transformations that preserve diagnostic value without requiring costly dual-energy CT hardware.

While the architecture described above primarily shows feature conditioning vectors adjusting features in the decoder pathway, in some embodiments, feature conditioning vectors may also be used to adjust feature maps continuing through the encoder pathway, so that the remainder of the encoding pathway processes adjusted feature maps. Furthermore, although an encoder-decoder neural network architecture is described in detail, the mechanism of conditioning feature maps using feature conditioning vectors can be applied to almost any neural network architecture, including but not limited to U-Net, residual dense networks (RDN), transformer-based architectures, and other convolutional neural network designs.

Referring to FIG. 5, a feature attention generator architecture 500 for generating a feature conditioning vector is shown. The architecture includes two parallel processing paths that converge to generate a feature conditioning vector used for dynamically adjusting features in energy-to-energy conversion of CT images. In one embodiment, the feature attention generator architecture 500 processes input priors including energy spectra and phase information and target priors including target energy spectra to generate feature conditioning vectors that control feature adjustment at decoder levels.

The first processing path begins with input priors 502 that characterize properties of an input CT image. In one embodiment, the input priors 502 include an input energy spectrum corresponding to a first energy level of the input CT image, providing X-ray attenuation values across an energy range. In another embodiment, the input priors 502 further include an input contrast phase timeline indicating temporal contrast agent dynamics, a display field of view (DFOV) value, and a reconstruction kernel type used to reconstruct the input CT image. The input priors 502 are processed through a first multi-layer perceptron 504 to generate first processed priors. The first multi-layer perceptron 504 may include multiple fully connected neural network layers with non-linear activation functions to learn representations of the input image characteristics that influence the energy conversion process.

The second processing path begins with target priors 506 that specify desired properties for the transformed CT image. In one embodiment, the target priors 506 include a target energy spectrum corresponding to a target energy level, providing desired X-ray attenuation characteristics. In another embodiment, the target priors 506 further include a target contrast phase timeline matching the input timeline and a target DFOV matching the input DFOV. The target priors 506 are processed through a second multi-layer perceptron 508 to generate second processed priors. Similar to the first multi-layer perceptron 504, the second multi-layer perceptron 508 learns representations of the target image characteristics to guide the transformation.

The outputs from both processing paths; the first processed priors from multi-layer perceptron 504 and the second processed priors from multi-layer perceptron 508, are combined to form combined processed features. In one embodiment, the outputs are concatenated to preserve distinct information from each path. The combined processed features are input to a third multi-layer perceptron 516 that learns relationships between the input characteristics and target characteristics. The third multi-layer perceptron 516 processes these relationships to generate a feature conditioning vector 518.

The feature conditioning vector 518 has a dimension corresponding to the number of channels in the feature map from the corresponding decoder level. In one embodiment, the feature conditioning vector 518 contains channel-wise conditioning factors ranging from 0 to 1 that are used to selectively adjust different feature channels through various operations. In another embodiment, the conditioning factors in the feature conditioning vector 518 are generated using a sigmoid activation function in the final layer of multi-layer perceptron 516 to ensure proper range. The feature conditioning vector 518 enables precise control over the transformation process by dynamically adjusting features based on the specific energy conversion requirements defined by the input and target priors.

This feature attention generator architecture 500 may be replicated at multiple decoder levels in the energy-to-energy neural network, with each instance processing the same input and target priors. The multiple feature conditioning vectors generated by these replicated architectures work together to maintain fine anatomical details and structural consistency while performing energy conversion between different energy levels and across different contrast phases.

A technical effect of the feature attention generator architecture 500 is that it enables precise control over feature adjustment at multiple network depths based on global energy transformation requirements. By incorporating input priors 502 including continuous X-ray energy spectra rather than discrete energy categories, the architecture can effectively interpolate or extrapolate to generate transformed images at any target energy level, even those not explicitly represented in the training data. The multi-layer perceptrons 504, 508, and 516 learn complex non-linear relationships between the input energy spectra and target energy spectra, allowing the network to dynamically adjust its behavior based on the specific energy conversion requirements. Additionally, by processing both input and target priors through dedicated pathways before combining them, the architecture maintains awareness of both the source and desired target characteristics throughout the transformation process. The generated conditioning vectors 518 provide factors that adjust different feature channels based on their relevance to the desired energy transformation, enabling the network to maintain fine anatomical details and structural consistency even when converting between energy levels that are far apart. The physics-based guidance from the energy spectra priors allows the network to learn generalizable relationships between energy levels rather than memorizing discrete mappings, making the architecture more robust and adaptable to novel energy conversion scenarios.

Referring to FIG. 6, a flowchart of a method 600 for transforming CT images between energy levels using a neural network with feature attention is shown. The method 600 enables transformation of CT images acquired at one energy level to appear as if acquired at a different energy level while maintaining image quality and anatomical detail. The method 600 may be carried out by an image processing system, such as image processing system 300 of FIG. 3.

At operation 602, the image processing system receives a CT image acquired at a first energy level. In one embodiment, the CT image may be acquired during an imaging protocol at 120 kVp, which produces X-ray spectra approximately equivalent to 70 keV monochromatic X-rays. In another embodiment, the CT image may be a maximum intensity projection (MIP) image reconstructed from helical scan projection data acquired during a contrast-enhanced imaging protocol.

At operation 604, the image processing system obtains input priors including an input energy spectrum corresponding to the first energy level. In one embodiment, the input energy spectrum is selected from a pre-recorded library of empirically measured spectra stored in energy spectra database 316 by matching acquisition parameters of the input CT image, including kVp setting, X-ray tube target material, and beam filtration, to corresponding parameters associated with each stored spectrum. In another embodiment, the input energy spectrum is determined by analyzing image metadata including scan protocol information to identify the acquisition energy level and corresponding spectrum. The input priors further include an input contrast phase timeline indicating temporal contrast agent dynamics derived from phase labeling by radiologists, a display field of view (DFOV) value used during image reconstruction, and a reconstruction kernel type indicating filtering applied during image reconstruction. In some embodiments, the input priors additionally include scan-specific parameters such as tube current, exposure time, and detector calibration data used during image acquisition. In other embodiments, the input priors may include geometric factors such as source-to-detector distance and detector element dimensions that influence spatial resolution characteristics.

At operation 606, the image processing system obtains target priors including a target energy spectrum corresponding to a target energy level. In one embodiment, the target energy spectrum is selected from a pre-recorded library of virtual monochromatic spectra stored in energy spectra database 316, with each spectrum representing narrow-band X-ray energies from 40 keV to 140 keV generated through physics-based modeling of X-ray interaction with matter. In another embodiment, the target energy spectrum is derived from empirical measurements of beam attenuation through calibration phantoms at different energy levels using dual-energy CT systems. The target priors further include a target contrast phase timeline matching the input timeline to maintain temporal consistency, a target DFOV matching the input DFOV to preserve spatial characteristics, and the reconstruction kernel type used for the input image to maintain consistent filtering effects. In some embodiments, the target priors additionally include noise and resolution characteristics expected at the target energy level based on system modeling. In other embodiments, the target priors may include ideal material decomposition bases representing expected attenuation behaviors of different tissue types at the target energy level.

At operation 608, the image processing system processes the CT image through an encoder pathway of a neural network to generate at least one feature map. In one embodiment, the encoder pathway includes multiple encoding levels operating at progressively reduced spatial resolutions, with each level having an associated feature attention generator to enable precise control over feature emphasis at every scale of the transformation process. In another embodiment, feature attention generators are selectively incorporated at specific encoder levels based on the magnitude of the energy level transformation being performed, with larger energy level changes (e.g., 70keV to 40 keV) utilizing more feature attention generators to maintain transformation accuracy, while smaller energy level changes may require fewer feature attention generators to balance computational efficiency. In yet another embodiment, feature attention generators are primarily incorporated at intermediate encoder levels where the spatial resolution and feature channel dimensions provide a balance between computational cost and transformation control, with early encoder levels focusing on extracting general image features and later encoder levels maintaining global context without requiring additional attention mechanisms. The encoder pathway may include 3-6 encoding levels depending on the input image resolution and desired feature hierarchy depth, with each level applying convolutional operations followed by pooling to reduce spatial dimensions while increasing feature channels, enabling the network to capture both fine anatomical details at higher resolutions and global contextual information at lower resolutions. The specific configuration of feature attention generators across encoder levels may be determined during network architecture design based on empirical evaluation of transformation quality versus computational overhead for different types of energy level conversions and contrast phases.

At operation 610, the image processing system generates a feature conditioning vector using a feature attention generator based on the input priors and target priors. In one embodiment, the feature attention generator processes the input and target energy spectra through parallel multilayer perceptrons (MLPs) with 3 hidden layers of 128, 256, and 128 nodes respectively, and generates channel-wise conditioning factors through a final MLP. In another embodiment, the feature attention generator may incorporate anatomical priors through an additional branch processing segmentation masks of key anatomical structures such as bones, soft tissue, and contrast-enhanced vessels. In yet another embodiment, the feature attention generator includes separate processing paths for contrast phase timelines and DFOV information, with dedicated MLPs having 2-4 hidden layers to learn relationships between these parameters and desired feature conditioning. In some embodiments, the feature attention generator first processes the input energy spectrum through an MLP with layer sizes [64, 128, 64], processes the target energy spectrum through a parallel MLP with layer sizes [64, 128, 64], concatenates these outputs, and processes through a final MLP with layer sizes [512, 256, C] where C is the number of feature channels, with batch normalization and ReLU activation between layers except for the final sigmoid activation.

At operation 612, the image processing system adjusts features of the feature map using the feature conditioning vector. In one embodiment, the conditioning vector contains channel-specific factors between 0 and 1 generated using a sigmoid activation function to selectively modify different feature channels based on their relevance to the target energy level transformation. In another embodiment, the conditioning factors may be spatially varying maps allowing different regions to be adjusted independently based on local image characteristics such as tissue type, contrast enhancement level, and anatomical location. In a further embodiment, the feature conditioning may be implemented as a combination of channel-wise and spatial conditioning, where each channel is first modified globally by the conditioning vector, followed by element-wise operations with a spatial conditioning map that modulates features based on local context. In one embodiment, the spatial conditioning maps are generated by processing local 3×3 neighborhoods of the feature maps through 1×1 convolutions followed by sigmoid activation, producing pixel-wise conditioning factors that can modify features based on both their channel identity and spatial context. The spatially-aware conditioning allows the network to adapt its feature transformation behavior based on local anatomical structures and their specific energy-dependent attenuation characteristics.

At operation 614, the image processing system processes the adjusted features through a decoder pathway of the neural network to generate a transformed CT image. In one embodiment, the decoder pathway includes multiple decoder levels corresponding to the number of encoder levels, where each decoder level uses transposed convolution operations to progressively upsample features while reducing channels, with skip connections from corresponding encoder levels enabling direct access to spatial details that may have been lost during encoding. In another embodiment, the number of decoder levels may be dynamically determined based on the input image resolution and desired feature reconstruction hierarchy, mirroring the dynamic level determination of the encoder pathway. In a further embodiment, each decoder level applies residual blocks with dual 3×3 convolutions to refine features before upsampling to the next resolution level, with the specific number and configuration of convolution operations matched to maintain symmetry with the corresponding encoder level. The decoder pathway may incorporate feature attention generators at some or all levels to enable selective feature conditioning during reconstruction, with the decision to include attention mechanisms at specific levels based on empirical evaluation of reconstruction quality versus computational cost for different types of energy conversions. In yet another embodiment, the decoder levels progressively reduce feature channels (e.g., from 512 to 256 to 128 to 64) while increasing spatial dimensions through transposed convolutions, with the specific channel progression matched to the corresponding encoder levels to maintain consistent feature representation capacity throughout the network. The decoder pathway may also incorporate additional skip connections within each level to facilitate gradient flow during training and enable learning of residual features relevant to energy-specific transformations, mirroring the residual connections in the encoder pathway.

At operation 616, the image processing system displays and/or saves the transformed CT image at the target energy level. In one embodiment, the transformed image is displayed alongside the original image to allow direct comparison of tissue contrast and anatomical detail between energy levels. In another embodiment, the transformed image is saved in DICOM format with appropriate tags indicating the synthetic nature of the image and the energy level transformation performed.

A technical effect of method 600 is that it enables accurate energy conversion of CT images while preserving anatomical detail through the coordinated use of physics-based priors and learned feature conditioning mechanisms. The multi-scale processing with selective feature modification through dynamically generated conditioning vectors at each decoder level allows the network to maintain high-frequency details while appropriately transforming contrast and tissue characteristics according to known X-ray physics principles. By incorporating both input and target energy spectra along with contrast phase timelines and reconstruction parameters as priors, the method enables precise control over energy conversion across different contrast phases and display fields of view. The feature attention generators, which may be selectively incorporated at specific encoder-decoder levels based on computational efficiency considerations, provide channel-wise and potentially spatially-varying conditioning factors that adapt the transformation behavior based on both local and global image characteristics. This unified approach eliminates the need for separate models for each energy combination and contrast phase while ensuring consistent quality across different anatomical regions, contrast enhancement patterns, and acquisition protocols.

Referring to FIG. 7, a flowchart of a method 700 for processing CT image data through multiple encoder levels is shown. The method 700 may be carried out by an image processing system, such as the image processing system 300 shown in FIG. 3, according to instructions stored in memory and executed by one or more processors. The method 700 may be performed as part of operation 608 of method 600 to process a CT image through an encoder pathway. The method 700 enables processing of CT images through a multi-level encoder pathway that incorporates feature conditioning mechanisms at each level to dynamically adjust features based on input and target priors. In one embodiment, the number of encoder levels may range from 3-6 depending on the input image resolution and desired feature hierarchy depth, with each level applying convolutional operations followed by pooling to reduce spatial dimensions while increasing feature channels.

At operation 702, the image processing system processes a CT image through a first encoder level, which includes several coordinated sub-operations that work together to extract and process meaningful features from the input image. At operation 704, the image processing system applies convolutional filters and activation functions to the CT image. In one embodiment, the convolution layer applies multiple learned convolutional filters of size 3×3 or 5×5 followed by non-linear activation functions such as ReLU to extract low-level image features including edges, textures, and intensity gradients characteristic of different tissue types and contrast-enhanced regions. In another embodiment, specialized kernels may be designed for capturing tissue boundaries, contrast-enhanced vessel structures, and localized energy-dependent attenuation patterns. At operation 706, the image processing system generates a first output encoder feature map based on the applied convolutional filters and activation functions. The feature map maintains structural and contrast information while beginning to capture energy-specific characteristics. At operation 708, the image processing system stores the output encoder feature map for use in skip connections with corresponding decoder levels, enabling direct access to earlier spatial information that may be lost during downsampling.

At operation 712, the image processing system determines if more encoder levels exist. If yes, the method proceeds to operation 714. If no, the method proceeds to operation 726. This decision point enables the network to process images through multiple encoding levels while maintaining precise control over feature extraction at each level. In one embodiment, the number of encoder levels may range from 3-6 depending on the input image resolution and desired feature hierarchy depth. The specific number of levels may be determined during network architecture design based on empirical evaluation of transformation quality versus computational overhead for different types of energy level conversions and contrast phases. In another embodiment, the decision to proceed through additional encoder levels may be dynamically determined based on the magnitude of the energy level transformation being performed, with larger energy level changes (e.g., 70 keV to 40 keV) utilizing more encoder levels to maintain transformation accuracy.

At operation 714, the image processing system downsamples the output encoder feature map for the next encoder level. In one embodiment, pooling operations using 2×2 windows and stride 2 reduce spatial dimensions while preserving dominant features such as tissue boundaries, contrast-enhanced vessel structures, and localized energy-dependent attenuation patterns. The downsampling helps achieve translation invariance and reduces computational complexity in deeper network layers. In another embodiment, the downsampling may be implemented through strided convolutions that learn downsampling patterns during training. The number of feature channels typically doubles at each successive encoder level (e.g., from 64 to 128 to 256) to maintain rich feature representations despite reduced spatial dimensions. This progressive increase in feature channels enables the network to capture increasingly complex patterns combining both local and global image characteristics while managing computational resources efficiently.

At operation 716, the image processing system processes the downsampled feature map through an ith encoder level. This includes several sub-operations: At operation 718, the image processing system applies convolutional filters and activation functions at the ith encoder level. In one embodiment, kernels of size 3×3 or 5×5 are designed for capturing intermediate-level features including tissue boundaries, contrast-enhanced vessel structures, and localized energy-dependent attenuation patterns characteristic of different anatomical regions. The convolutional filters may employ dilated convolutions with varying dilation rates (e.g., 2, 4, or 8) to capture multi-scale contextual information without increasing computational overhead. In another embodiment, the activation functions include LeakyReLU with a negative slope of 0.01 to prevent dying ReLU problems during training, or PReLU with learnable slopes for each channel. At operation 720, the image processing system generates an ith output encoder feature map containing more abstract representations than preceding levels, with the number of feature channels typically doubling from the previous level (e.g., from 128 to 256 channels) to maintain rich feature representations despite reduced spatial dimensions. At operation 722, the image processing system stores the ith output encoder feature map for skip connections to corresponding decoder levels, enabling direct access to intermediate features that help preserve fine anatomical details during upsampling. The skip connections may employ 1×1 convolutions to match channel dimensions before concatenation with decoder features.

At operation 726, the image processing system downsamples the Nth output encoder feature map and passes it to the first decoder level, where N represents the total number of encoder levels in the network architecture. The final encoder level operates at the lowest spatial resolution while maintaining rich feature representations through increased channel depth. In one embodiment, the lowest level operates at 1/16 or 1/32 of the original image resolution with 512 or 1024 feature channels, using max pooling or strided convolutions for downsampling. The increased channel depth at this level enables capture of highly abstract representations combining both local and global image characteristics relevant for energy conversion. In another embodiment, the final encoder level may incorporate residual connections or dense connections to facilitate gradient flow during training, with each residual block containing dual 3×3 convolutions followed by batch normalization and ReLU activation. The specific configuration of the lowest level, including resolution reduction factor and number of feature channels, may be determined during architecture design based on input image resolution and desired feature hierarchy depth, with larger images potentially benefiting from additional downsampling to achieve an appropriate balance between computational efficiency and feature representation capacity. After operation 726, the method 700 returns to continue with operation 610 of method 600.

Referring to FIG. 8, a flowchart of a method 800 for decoder-based CT image transformation is shown. The method 800 may be carried out by an image processing system, such as image processing system 300 of FIG. 3, to process feature maps through a decoder pathway and generate a transformed CT image at a target energy level. The method 800 may be performed as part of operation 614 of method 600 to process scaled features through a decoder pathway.

At operation 802, the image processing system processes a first decoder level through several coordinated sub-operations. At operation 804, the image processing system receives an Nth encoder feature map from the lowest level of the encoder pathway, which contains abstract representations combining both local and global image characteristics relevant for energy conversion. In one embodiment, the Nth encoder feature map operates at 1/16 or 1/32 of the original image resolution with 512 or 1024 feature channels to maintain rich representational capacity despite reduced spatial dimensions. In another embodiment, the Nth encoder feature map includes residual connections to facilitate gradient flow during training, with each residual block containing dual 3×3 convolutions followed by batch normalization and ReLU activation. At operation 806, the image processing system applies convolutional filters and activation functions to the Nth encoder feature map. In one embodiment, multiple learned convolutional filters of size 3×3 or 5×5 are applied followed by non-linear activation functions such as ReLU to process the feature map, with specialized kernels designed for capturing tissue boundaries, contrast-enhanced vessel structures, and energy-dependent attenuation patterns. In another embodiment, the convolution operations employ dilated convolutions with varying dilation rates to capture multi-scale contextual information without increasing computational overhead.

At operation 808, the image processing system generates an output decoder feature map based on the applied convolutional filters and activation functions. The output decoder feature map maintains structural and contrast information while beginning to capture energy-specific characteristics through the coordinated application of learned filters and non-linear transformations. In one embodiment, the output decoder feature map maintains the same channel dimensionality as the input while refining the feature representations through the applied transformations. In another embodiment, additional post-processing operations such as channel attention or adaptive normalization may be applied to enhance the quality of the output feature map.

At operation 810, the image processing system determines if more decoder levels exist. This decision point enables the network to process features through multiple decoding levels while maintaining precise control over feature reconstruction at each level. The specific number of decoder levels may be determined during network architecture design based on empirical evaluation of transformation quality versus computational overhead for different types of energy level conversions and contrast phases. In one embodiment, the number of decoder levels matches the number of encoder levels, and may range from 4-6 levels depending on the input image resolution and desired feature hierarchy depth. In another embodiment, the decision to proceed through additional decoder levels may be dynamically determined based on the magnitude of the energy level transformation being performed, with larger energy level changes utilizing more decoder levels to maintain transformation accuracy.

If more decoder levels exist, at operation 812, the image processing system up-convolves the output decoder feature map for the next decoder level. In one embodiment, transposed convolution operations with learnable filters of size 2×2 or 4×4 and stride 2 are used to double the spatial dimensions while reducing the number of feature channels, typically halving the number of channels at each level to maintain computational efficiency while preserving salient features. In another embodiment, the up-convolution may employ different interpolation methods such as bilinear upsampling followed by standard convolutions, with the specific approach chosen based on empirical evaluation of reconstruction quality and computational requirements. The up-convolution operations are designed to minimize checkerboard artifacts while maintaining smooth feature reconstruction across different spatial scales.

At operation 814, the image processing system processes the up-convolved feature map through an ith decoder level through several coordinated sub-operations. At operation 816, the image processing system concatenates the up-convolved feature map with the output encoder feature map from the corresponding encoder level through skip connections. This enables direct access to earlier spatial information that may have been lost during encoding, helping preserve fine anatomical details and structural consistency. In one embodiment, 1×1 convolutions may be applied before concatenation to match channel dimensions and learn feature combinations. At operation 818, the image processing system generates a feature conditioning vector using an ith feature attention generator based on input priors including energy spectra and phase information, and target priors.

At operation 820, the image processing system adjusts the concatenated feature map using the ith feature conditioning vector. In one embodiment, each channel of the feature map is modified by corresponding values in the feature conditioning vector, which may involve scaling, shifting, or other transformations to selectively modify different feature channels based on their relevance to the target energy level. In another embodiment, the adjustment may be implemented as a combination of channel-wise and spatial conditioning, where each channel is first modified globally by the conditioning vector, followed by element-wise operations with a spatial conditioning map that modulates features based on local context. At operation 822, the image processing system applies convolutional filters and activation functions to the adjusted features. In one embodiment, multiple 3×3 convolutional filters followed by batch normalization and ReLU activation are applied to process the adjusted features, with the specific number and configuration of convolution operations matched to maintain symmetry with the corresponding encoder level. At operation 824, the image processing system generates an ith output decoder feature map containing refined local details while maintaining energy-specific characteristics.

If no more decoder levels exist at operation 810, method 800 proceeds to operation 826. At operation 826, the image processing system applies final convolutional filters and activation functions to produce a transformed CT image at the target energy level. In one embodiment, a final set of convolutional filters (e.g., 32 or 64 3×3 filters) followed by a 1×1 convolution generates the output image, with the specific filter configurations designed to enhance the final reconstruction quality while maintaining computational efficiency. In another embodiment, additional post-processing operations such as residual connections or adaptive normalization may be applied to enhance the final output quality, with the specific enhancements chosen based on empirical evaluation of image quality metrics and clinical requirements. The transformed image maintains both fine anatomical details and accurate energy-specific characteristics through the coordinated use of multi-scale processing, feature attention mechanisms, and specialized convolutional operations throughout the decoder pathway.

Referring to FIG. 9, a flowchart of a method 900 for generating a feature conditioning vector for a decoder level is shown. The method 900 may be carried out by an image processing system, such as image processing system 300 of FIG. 3, as part of operation 610 of method 600 to generate feature conditioning vectors that enable precise control over feature adjustment during energy-to-energy conversion of CT images. The feature attention generators enable a single unified network to handle conversion between arbitrary energy levels by incorporating continuous energy spectra as priors for both input and target images, allowing the network to learn a shared manifold for energy-to-energy transformations while maintaining fine anatomical details and structural consistency across different contrast phases. This approach is both computationally efficient, requiring only one model rather than separate models for each energy level combination, and robust due to the network's ability to leverage a larger combined training dataset spanning multiple energy levels and contrast phases.

At operation 908, the image processing system processes input priors through a first MLP to generate processed input priors. In one embodiment, the input priors include an input energy spectrum corresponding to the first energy level, providing X-ray attenuation values across an energy range, along with an input contrast phase timeline indicating temporal contrast agent dynamics and display field of view values used during image reconstruction. The first MLP processes these priors through multiple fully connected layers to learn representations that influence the energy conversion process, enabling the network to adapt its behavior based on acquisition parameters. In another embodiment, the input priors may additionally include reconstruction kernel types and scan-specific parameters such as tube current and exposure time that affect image characteristics.

At operation 910, the image processing system processes target priors through a second MLP to generate processed target priors. In one embodiment, the target priors include a target energy spectrum corresponding to the target energy level and a target contrast phase timeline matching the input timeline to maintain temporal consistency. The second MLP has an architecture similar to the first MLP but with independent weights, allowing it to learn specific representations for the desired target characteristics while maintaining architectural symmetry. In another embodiment, the target priors may include ideal material decomposition bases representing expected attenuation behaviors of different tissue types at the target energy level.

At operation 912, the image processing system concatenates the outputs of the first MLP and second MLP to form combined processed features. In one embodiment, the concatenation operation preserves distinct information from each processing path by stacking the processed input priors and processed target priors into a single feature vector that maintains separate representations of input energy properties and target energy requirements. The combined features enable the network to consider multiple factors simultaneously when determining appropriate feature conditioning factors. In another embodiment, the concatenated features may undergo additional normalization or dimensionality adjustment operations to ensure balanced contribution from each information stream.

At operation 914, the image processing system processes the combined features through a third MLP to generate a feature conditioning vector for the ith decoder level. In one embodiment, the third MLP outputs a vector with dimension matching the number of channels in the corresponding decoder level feature map, with values normalized between 0 and 1 using a sigmoid activation function to provide appropriate conditioning factors for selective feature adjustment. The feature conditioning vector enables channel-wise adjustment of decoder features through various operations, allowing the network to selectively modify different feature channels based on the specific energy conversion requirements. In another embodiment, the third MLP may incorporate residual connections to facilitate gradient flow during training, with each residual block containing dual fully connected layers followed by batch normalization and ReLU activation.

Referring to FIG. 10, a flowchart of a method 1000 for training a neural network to transform medical images between different energy levels is shown. The method 1000 may be carried out by an image processing system, such as image processing system 300 of FIG. 3, to train the energy-to-energy neural network 308 to transform CT images from one energy level to another energy level while maintaining image quality across different contrast phases and display fields of view. The method 1000 enables training of a unified energy-to-energy neural network that can perform multiple energy-level conversions rather than requiring separate models for each energy combination and contrast phase.

At operation 1002, the image processing system receives a plurality of training image pairs, each pair including an input image at a first energy level and a target image at a second energy level. In one embodiment, the input image and target image are monochromatic images generated from dual-energy projection data, with the input image acquired at the first energy level (e.g., 70 keV) and the target image acquired at the second energy level (e.g., 40keV, 50 keV, or 60 keV). In another embodiment, the training image pairs may include images from various contrast phases (e.g., CTPA, CTA, arterial, portal, delayed, and delayed-equilibrium phases) and display fields of view (DFOV) ranging from 29-50 cm, enabling the neural network to learn transformations that are robust across different imaging conditions. The training image pairs may be pre-labeled by radiologists to indicate their respective contrast phases, allowing the network to learn phase-specific transformation characteristics.

At operation 1004, the image processing system obtains input priors including input energy spectra corresponding to the first energy level and input contrast phase timeline. In one embodiment, the input energy spectra are selected from a pre-recorded library of empirically measured spectra stored in energy spectra database 316, providing X-ray attenuation values across an energy range corresponding to the first energy level. In another embodiment, the input priors further include an input contrast phase timeline indicating temporal contrast agent dynamics, a display field of view (DFOV) value used during image reconstruction, and a reconstruction kernel type indicating filtering applied during image reconstruction. These input priors provide physics-based guidance to the neural network during training, enabling it to learn energy-specific transformations that account for both image content and acquisition parameters.

At operation 1006, the image processing system obtains target priors including target energy spectra corresponding to the second energy level and target contrast phase timeline. In one embodiment, the target energy spectra are selected from a pre-recorded library of virtual monochromatic spectra stored in energy spectra database 316, with each spectrum representing narrow-band X-ray energies from 40 keV to 140 keV. In another embodiment, the target priors further include a target contrast phase timeline matching the input timeline to maintain temporal consistency, a target DFOV matching the input DFOV to preserve spatial characteristics, and the reconstruction kernel type used for the input image. These target priors define the desired characteristics of the transformed images, guiding the network to produce outputs that accurately reflect the expected appearance at the target energy level.

At operation 1008, the image processing system processes the input image through an encoder pathway of the neural network to generate feature maps. In one embodiment, the encoder pathway includes multiple encoding levels operating at progressively reduced spatial resolutions, with each level applying convolutional operations followed by pooling to reduce spatial dimensions while increasing feature channels. In another embodiment, the encoder pathway may include 3-6 encoding levels depending on the input image resolution and desired feature hierarchy depth, with each level extracting increasingly abstract representations combining both local and global image characteristics relevant for energy conversion. The encoder pathway enables the network to capture both fine anatomical details at higher resolutions and global contextual information at lower resolutions.

At operation 1010, the image processing system generates feature conditioning vectors using feature attention generators based on input priors and target priors. In one embodiment, each feature attention generator processes the input and target energy spectra through parallel MLPs and generates channel-wise conditioning factors through a final MLP. In another embodiment, the feature attention generators may be incorporated at multiple encoder-decoder levels to enable precise control over feature adjustment at every scale of the transformation process. The feature conditioning vectors enable the network to dynamically adjust its behavior based on global target energy characteristics, allowing it to selectively modify different feature channels based on their relevance to the target energy level.

At operation 1012, the image processing system adjusts features of the feature maps using the feature conditioning vectors. In one embodiment, the conditioning vectors contain channel-specific factors between 0 and 1 generated using a sigmoid activation function to selectively modify different feature channels. In another embodiment, the adjustment may be implemented as a combination of channel-wise and spatial conditioning, where each channel is first modified globally by the conditioning vector, followed by element-wise operations with a spatial conditioning map that modulates features based on local context. This selective feature adjustment enables the network to adapt its transformation behavior based on the specific energy conversion requirements.

At operation 1014, the image processing system processes the adjusted features through a decoder pathway to generate a transformed image at the second energy level. In one embodiment, the decoder pathway includes multiple decoder levels corresponding to the number of encoder levels, where each decoder level uses transposed convolution operations to progressively upsample features while reducing channels, with skip connections from corresponding encoder levels enabling direct access to spatial details that may have been lost during encoding. In another embodiment, each decoder level applies residual blocks with dual 3×3 convolutions to refine features before upsampling to the next resolution level, with the specific number and configuration of convolution operations matched to maintain symmetry with the corresponding encoder level. The decoder pathway reconstructs the transformed image at the target energy level while preserving fine anatomical details and structural consistency.

At operation 1016, the image processing system compares the transformed image with the target image and adjusts neural network parameters via backpropagation. In one embodiment, a loss is determined based on the difference between the transformed image and the target image, with a loss function used to update the parameters of the neural network through backpropagation. In another embodiment, the loss function may include multiple components such as a pixel-wise mean squared error, a perceptual loss term based on feature differences in a pre-trained network, and a structural similarity term to ensure preservation of anatomical structures. In one embodiment, the loss function may specifically incorporate energy-dependent weighting factors that emphasize regions where energy-specific contrast differences are most pronounced, such as contrast-enhanced vessels and organ boundaries. In yet another embodiment, the loss function may include a cyclic consistency term that ensures the transformed image can be accurately converted back to the original energy level through an inverse transformation, preserving structural integrity and discouraging tissue or contrast mixing across voxels. This forward-backward consistency is particularly important for maintaining diagnostic quality across different energy levels and contrast phases. The loss function may further include phase-specific components that adapt based on the contrast phase timeline provided as prior information, with higher weights assigned to anatomical regions expected to show significant enhancement during specific contrast phases. For example, during arterial phase transformations, the loss function may place greater emphasis on accurately preserving contrast differences in arterial structures, while during portal phase transformations, greater emphasis may be placed on liver parenchyma and portal vessels. In some embodiments, the loss function may also incorporate a physics-based constraint derived from the input and target energy spectra to ensure the transformed image reflects expected attenuation changes based on known X-ray physics principles.

The backpropagation process enables the network to iteratively improve its transformation accuracy by adjusting weights and biases to minimize the difference between transformed images and ground truth target images. For the feature attention generators, the backpropagation process specifically updates the parameters of the multiple MLPs that process the energy spectra and phase information, allowing the network to learn feature conditioning factors for different energy level combinations and contrast phases. This targeted parameter adjustment ensures the network can generalize to energy level combinations and contrast phases not explicitly represented in the training data, enabling a single unified model to handle multiple energy conversion tasks that would otherwise require separate specialized models.

By incorporating both input and target energy spectra along with contrast phase timelines and reconstruction parameters as priors during the training process, method 1000 enables the neural network to learn physics-based relationships between energy levels rather than memorizing discrete mappings. The feature conditioning mechanisms trained through operations 1010-1012 provide channel-specific and potentially spatially-varying conditioning factors that adapt transformation behavior based on global image characteristics. The backpropagation process at operation 1016 specifically updates the parameters of the multiple MLPs that process the energy spectra and phase information, allowing the network to learn feature conditioning factors for different energy level combinations and contrast phases. This targeted parameter adjustment ensures the network can generalize to energy level combinations and contrast phases not explicitly represented in the training data, enabling a single unified model to handle multiple energy conversion tasks that would otherwise require separate specialized models. Additionally, the cyclic consistency loss component incorporated in operation 1016 ensures the transformed image can be accurately converted back to the original energy level through an inverse transformation, preserving structural integrity and discouraging tissue or contrast mixing across voxels. The training approach of method 1000 significantly reduces computational overhead and system complexity while ensuring consistent quality across different anatomical regions, contrast enhancement patterns, and acquisition protocols.

Referring to FIG. 11, a flowchart of a method 1100 for training a neural network to transform medical images between different energy levels is shown. The method 1100 may be carried out by an image processing system, such as image processing system 300 of FIG. 3, to train the energy-to-energy neural network 308 to transform CT images from one energy level to multiple target energy levels while maintaining image quality across different contrast phases and display fields of view. The method 1100 enables training of a unified energy-to-energy neural network that can perform multiple energy-level conversions rather than requiring separate models for each energy combination and contrast phase.

At operation 1102, the image processing system receives a training image set including an input image at a first energy level and multiple target images at different energy levels generated from dual-energy projection data. In one embodiment, the input image and target images are monochromatic images generated from dual-energy projection data, with the input image acquired at the first energy level (e.g., 70 keV) and the target images acquired at different target energy levels (e.g., 40keV, 50 keV, and 60 keV). In another embodiment, the training image set may include images from various contrast phases (e.g., CTPA, CTA, arterial, portal, delayed, and delayed-equilibrium phases) and display fields of view (DFOV) ranging from 29-50 cm, enabling the neural network to learn transformations that are robust across different imaging conditions. The training image sets may be pre-labeled by radiologists to indicate their respective contrast phases, allowing the network to learn phase-specific transformation characteristics.

At operation 1104, the image processing system obtains input priors including input energy spectra corresponding to the first energy level and input contrast phase timeline. In one embodiment, the input energy spectra are selected from a pre-recorded library of empirically measured spectra stored in energy spectra database 316, providing X-ray attenuation values across an energy range corresponding to the first energy level. In another embodiment, the input priors further include a display field of view (DFOV) value used during image reconstruction, and a reconstruction kernel type indicating filtering applied during image reconstruction. These input priors provide physics-based guidance to the neural network during training, enabling it to learn energy-specific transformations that account for both image content and acquisition parameters.

At operation 1106, the image processing system obtains target priors including target energy spectra corresponding to the target energy levels and target contrast phase timeline for each target energy level. In one embodiment, the target energy spectra are selected from a pre-recorded library of virtual monochromatic spectra stored in energy spectra database 316, with each spectrum representing narrow-band X-ray energies from 40 keV to 140 keV. In another embodiment, the target priors further include a target contrast phase timeline matching the input timeline to maintain temporal consistency, a target DFOV matching the input DFOV to preserve spatial characteristics, and the reconstruction kernel type used for the input image. These target priors define the desired characteristics of the transformed images, guiding the network to produce outputs that accurately reflect the expected appearance at each target energy level.

At operation 1108, the image processing system processes the input image through a neural network using input priors and target priors to map the input image to each target energy level, including an identity transformation at the input energy level. In one embodiment, the neural network processes the input image through an encoder pathway to generate feature maps, generates feature conditioning vectors using feature attention generators based on input priors and target priors, adjusts features of the feature maps using the feature conditioning vectors, and processes the adjusted features through a decoder pathway to generate transformed images at each target energy level. The identity transformation at the input energy level serves as an additional constraint during training, ensuring that when the target energy level matches the input energy level, the network learns to preserve the original image characteristics without introducing artifacts or distortions.

At operation 1110, the image processing system compares each transformed image with a corresponding target image at a respective target energy level, including comparing the identity transformed image with the input image. In one embodiment, the comparison includes calculating multiple similarity metrics between each transformed image and its corresponding target image, such as mean squared error, structural similarity index, and perceptual loss based on feature differences in pre-trained networks. In another embodiment, the comparison may incorporate energy-dependent weighting factors that emphasize regions where energy-specific contrast differences are most pronounced, such as contrast-enhanced vessels and organ boundaries. The comparison of the identity transformed image with the input image serves as a self-consistency check, ensuring the network maintains image fidelity even when no energy transformation is required.

At operation 1112, the image processing system aggregates loss from all comparisons and adjusts neural network parameters via backpropagation. In one embodiment, the aggregated loss function combines individual losses from each energy level transformation with different weighting factors based on the magnitude of energy level change, with, in some embodiments, larger energy level transformations receiving higher weights to ensure transformation fidelity across larger energy differences between input and target. In another embodiment, the loss function may include a cyclic consistency term that ensures transformed images can be accurately converted back to the original energy level through an inverse transformation, preserving structural integrity and discouraging tissue or contrast mixing across voxels. This multi-target training approach enables the network to learn a shared manifold for energy-to-energy transformations while maintaining fine anatomical details and structural consistency across different contrast phases.

At operation 1114, the image processing system stores the trained neural network model. In one embodiment, the trained model is stored in non-transitory memory 306 as energy-to-energy neural network 308 for subsequent use in transforming clinical CT images between different energy levels. In another embodiment, the stored model includes not only the network weights and architecture but also the feature attention generators and prior encoding modules that enable the network to process energy spectra and phase timeline information during inference. The stored model provides a unified solution for energy conversion across different contrast phases and display fields of view, eliminating the need for separate models for each energy combination and contrast phase.

A technical effect of method 1100 is that it enables training of a single unified neural network capable of performing multiple energy-level conversions while maintaining consistency across different contrast phases and display fields of view. By training with multiple target energy levels simultaneously and incorporating both input and target energy spectra along with contrast phase timelines as priors, the method enables the network to learn physics-based relationships between energy levels rather than memorizing discrete mappings. The multi-target training approach with aggregated loss significantly reduces computational overhead and system complexity while ensuring consistent quality across different anatomical regions, contrast enhancement patterns, and acquisition protocols. The inclusion of identity transformations in the training process further enhances the network's ability to maintain image fidelity across the full spectrum of energy transformations.

Referring to FIG. 12, a graphical representation of an X-ray spectrum 1200 showing the relationship between spectral intensity and voltage at various operating voltages is shown. The X-ray spectrum 1200 includes a voltage axis 1202 ranging from 0 to 150 kilovolts and a spectral amplitude axis 1204 ranging from 0 to 14 × 106 in arbitrary units. The X-ray spectrum 1200 illustrates how X-ray intensity varies with energy, which serves as the physical basis for the input and target priors used by the energy-to-energy neural network described herein.

The X-ray spectrum 1200 displays multiple spectral curves corresponding to different peak kilovoltage (kVp) settings commonly used in CT imaging, including a 70 kVp curve 1206, an 80 kVp curve 1208, a 90 kVp curve 1210, a 100kVp curve 1212, a 110 kVp curve 1214, a 120 kVp curve 1216, a 130kVp curve 1218, and a 140 kVp curve 1220. In one embodiment, these spectral curves represent the energy spectra stored in the energy spectra database 316 of FIG. 3, which are provided as input priors and target priors to the feature attention generators 452 and 456 shown in FIG. 4. In another embodiment, the spectral curves represent the X-ray intensity values across energy ranges that are encoded by the prior encoding module 312 before being processed by the feature attention generators.

Each spectral curve exhibits a primary characteristic peak 1222 and a secondary characteristic peak 1224, which are distinctive features resulting from the X-ray tube target material. In one embodiment, the primary characteristic peak 1222 corresponds to the K-alpha emission line of the target material, while the secondary characteristic peak 1224 corresponds to the K-beta emission line. In another embodiment, these characteristic peaks provide unique spectral signatures that enable the feature attention generators to distinguish between different energy levels when transforming images. The position and amplitude of the primary characteristic peak 1222 and secondary characteristic peak 1224 vary with different kVp settings, providing energy-specific information that guides the transformation process. This energy-specific information enables the energy-to-energy neural network 308 to accurately transform images between different energy levels.

The spectral curves illustrate that with increasing kVp, X-ray intensity generally increases across the energy spectrum, with the peak intensity shifting toward higher energies. This physical relationship forms the basis for the contrast differences observed when transforming images between different energy levels (e.g., from 70 keV to 50 keV or 40 keV). By providing these energy spectra as input priors and target priors to the feature attention generators, the energy-to-energy neural network can accurately model the energy-dependent characteristics of different tissues and contrast agents, enabling precise energy conversion while maintaining image quality across different contrast phases and display fields of view.

Referring to FIG. 13, examples of encoded phase contrast timelines are shown in a timing diagram 1300 that illustrates how contrast phase information is encoded as vectors for use in the energy-to-energy neural network. The timing diagram 1300 includes multiple sequences representing different contrast phases, with each sequence encoded as a vector of predetermined length where a subset of consecutive elements are set to non-zero values (represented as ones) corresponding to a time period of the indicated contrast phase, while remaining elements are set to zero.

Sequence 1302 shows activation in the delayed equilibrium phase, where non-zero values appear at the rightmost portion of the vector, indicating that contrast agent has reached the delayed equilibrium state where contrast exits the body. Sequence 1304 shows activation in the delayed phase, with non-zero values positioned earlier than sequence 1302 but later than other sequences, representing the period when contrast has diffused into interstitial spaces. Sequence 1306 shows activation in the portal phase, with non-zero values positioned in the middle portion of the vector, corresponding to the time when contrast enhances the portal and hepatic veins. Sequence 1308 shows activation in the arterial phase, with non-zero values positioned earlier in the timeline, representing the period when contrast primarily enhances arterial structures.

The time axis phases progress from left to right, beginning with non-contrast (before contrast injection), followed by CTPA (pulmonary artery phase), aortic (CTA), late arterial (arterial), portal, delayed, and delayed equilibrium phases. This progression corresponds to the physiological timeline of contrast agent flow through the body after injection.

The phase timeline module 314 maintains this phase timeline data indicating contrast enhancement patterns across different anatomical structures over time, providing phase timeline priors to enable the network to maintain consistency across different contrast phases during energy conversion.

These encoded phase contrast timelines may be incorporated into prior information 450 that guides the feature attention generators in dynamically adjusting features based on the specific contrast phase of the input image, enabling accurate energy conversion across the full contrast phase cycle.

Referring to FIG. 14, a comparative display of CT images showing input, target, and predicted images at different energy levels (70keV, 60keV, and 50keV) is shown. The display includes a top row of reference images and a bottom row of predicted images generated using the unified energy-to-energy neural network described herein. The comparative display enables visual assessment of the herein disclosed energy-to-energy neural network's capability to transform CT images between different energy levels while maintaining anatomical detail and image quality across different contrast phases and display fields of view.

The top row includes an input 70keV image 1402, which in one embodiment is an original CT image acquired at 70keV (approximately equivalent to 120kVp) during a portal contrast phase. In another embodiment, the input 70keV image 1402 may be a virtual monochromatic image generated from dual-energy projection data. The input 70keV image 1402 serves as the source image for energy conversion and provides baseline anatomical information for comparison with the transformed images. Adjacent to the input image is a target 60keV image 1404, which in one embodiment is a ground truth image acquired at 60keV using dual-energy CT imaging techniques. In another embodiment, the target 60keV image 1404 may be a reference image generated through material decomposition of dual-energy projection data. The target 60keV image 1404 demonstrates the expected appearance and contrast characteristics at the 60keV energy level. Further to the right is a target 50keV image 1406, which in one embodiment is a ground truth image acquired at 50keV using dual-energy CT imaging techniques. In another embodiment, the target 50keV image 1406 may be a reference image generated through material decomposition of dual-energy projection data. The target 50keV image 1406 demonstrates the expected appearance and contrast characteristics at the 50keV energy level, showing enhanced contrast compared to the 60keV and 70keV images.

The bottom row includes a predicted 70keV image 1408, which in one embodiment is generated by the energy-to-energy neural network when provided with the input 70keV image 1402 and both input and target priors specifying 70keV as both the input and target energy level. In another embodiment, the predicted 70keV image 1408 may be generated as part of a cyclic consistency validation process to verify the network's ability to maintain image quality when the target energy matches the input energy. The predicted 70keV image 1408 should closely match the input 70keV image 1402, demonstrating the network's ability to preserve image characteristics when no energy transformation is required. Adjacent image 1408 is a predicted 60keV image 1410, which in one embodiment is generated by the energy-to-energy neural network when provided with the input 70keV image 1402, input priors specifying 70keV, and target priors specifying 60keV. The predicted 60keV image 1410 closely matches the target 60keV image 1404, demonstrating the network's ability to accurately transform images to intermediate energy levels. Further to the right is a predicted 50keV image 1412, which in one embodiment is generated by the energy-to-energy neural network when provided with the input 70keV image 1402, input priors specifying 70keV, and target priors specifying 50keV. The predicted 50keV image 1412 closely matches the target 50keV image 1406, demonstrating the network's ability to accurately transform images to lower energy levels with enhanced contrast visualization.

Referring to FIG. 15, a comparative visualization of the results of energy level conversion using the herein disclosed energy-to-energy neural network, is shown. The FIG. 15 includes three panels demonstrating the transformation from a higher energy (70keV) input image to a lower energy (50keV) predicted image, alongside the actual target image for comparison.

The input 70keV image 1502 is shown in the left panel of FIG. 15. In one embodiment, the input 70keV image 1502 is an original CT image acquired at 70keV during a portal contrast phase. In another embodiment, the input 70keV image 1502 may be a virtual monochromatic image generated from dual-energy projection data. The input 70keV image 1502 serves as the source image for energy conversion and provides baseline anatomical information for comparison with the transformed images. The input 70keV image 1502 demonstrates typical contrast characteristics at this energy level, with moderate tissue differentiation and contrast enhancement in vascular structures.

The predicted 50keV image 1504 is shown in the middle panel of FIG. 15. In one embodiment, the predicted 50keV image 1504 is generated by the energy-to-energy neural network when provided with the input 70keV image 1502, input priors specifying 70keV energy spectrum, and target priors specifying 50keV energy spectrum. The predicted 50keV image 1504 demonstrates enhanced contrast compared to the input 70keV image 1502, particularly in contrast-enhanced regions.

The target 50keV image 1506 is shown in the right panel of FIG. 15. In one embodiment, the target 50keV image 1506 is a ground truth image acquired at 50keV using dual-energy CT imaging techniques. In another embodiment, the target 50keV image 1506 may be a reference image generated through material decomposition of dual-energy projection data. The target 50keV image 1506 demonstrates the expected appearance and contrast characteristics at the 50keV energy level, showing enhanced contrast compared to the 70keV image. The close visual similarity between the predicted 50keV image 1504 and the target 50keV image 1506 demonstrates the accuracy of the energy-to-energy neural network in performing the energy conversion while preserving anatomical details and enhancing contrast visualization.

The disclosure also provides support for a method, comprising: receiving a computed tomography (CT) image acquired at a first energy level, obtaining input priors including an input energy spectrum corresponding to the first energy level, obtaining target priors including a target energy spectrum corresponding to a target energy level, processing the CT image through a neural network to generate at least one feature map, generating, using a feature attention generator, a feature conditioning vector based on the input priors and the target priors, adjusting features of the at least one feature map using the feature conditioning vector to generate adjusted features, and processing the adjusted features through the neural network to generate a transformed CT image at the target energy level. In a first example of the method, the input priors further include: an input contrast phase timeline, and an input Display Field of View (DFOV). In a second example of the method, optionally including the first example, generating the feature conditioning vector comprises: processing the input priors through a first neural network to generate first processed priors, processing the target priors through a second neural network to generate second processed priors, concatenating the first processed priors and the second processed priors to form combined processed features, and processing the combined processed features through a third neural network to generate the feature conditioning vector, wherein the feature conditioning vector has a dimension corresponding to a number of channels in the at least one feature map. In a third example of the method, optionally including one or both of the first and second examples, adjusting features of the at least one feature map using the feature conditioning vector comprises modifying the at least one feature map through one or more operations based on numerical values of the feature conditioning vector. In a fourth example of the method, optionally including one or more or each of the first through third examples, the one or more operations include multiplying each channel of the at least one feature map by a corresponding scalar value in the feature conditioning vector.

The disclosure also provides support for a method, comprising: receiving a computed tomography (CT) image acquired at a first energy level, obtaining input priors including an input energy spectrum corresponding to the first energy level, obtaining target priors including a target energy spectrum corresponding to a target energy level, processing the CT image through a neural network to generate a transformed CT image at the target energy level, wherein the neural network comprises: an encoder pathway comprising a plurality of encoder levels, wherein each encoder level is configured to receive an input feature map and convolve the input feature map with one or more convolutional filters to generate an output encoder feature map, and a decoder pathway comprising a plurality of decoder levels corresponding to the plurality of encoder levels, wherein each decoder level is configured to receive an input decoder feature map comprising an up-convolved feature map from a lower decoder level concatenated with the output encoder feature map from a corresponding encoder level, generating at each decoder level, using a feature attention generator, a feature conditioning vector based on the input priors and the target priors, adjusting features of the input decoder feature map at each decoder level using a corresponding feature conditioning vector to generate adjusted features, convolving the adjusted features at each decoder level to generate an output decoder feature map, and outputting the transformed CT image based on an output decoder feature map from a highest level of the decoder pathway. In a first example of the method, generating the feature conditioning vector at each decoder level comprises: processing the input priors through a first MLP to generate processed input priors, processing the target priors through a second MLP to generate processed target priors, combining the processed input priors and processed target priors to generate combined features, and processing the combined features through a third MLP to generate the feature conditioning vector. In a second example of the method, optionally including the first example, combining the processed input priors, and the processed target priors comprises concatenating the processed input priors, and processed target priors. In a third example of the method, optionally including one or both of the first and second examples, adjusting features of the input decoder feature map comprises multiplying each channel of the input decoder feature map by a corresponding scalar value in the feature conditioning vector. In a fourth example of the method, optionally including one or more or each of the first through third examples, the input priors further comprise a contrast phase timeline indicating a contrast phase of the CT image, and wherein the target priors further comprise a target contrast phase timeline. In a fifth example of the method, optionally including one or more or each of the first through fourth examples, the contrast phase timeline comprises a one-dimensional curve indicating one or more of: a pulmonary artery phase, an aortic phase, a late arterial phase, a portal phase, a delayed phase, or a delayed equilibrium phase. In a sixth example of the method, optionally including one or more or each of the first through fifth examples, the contrast phase timeline is encoded as a vector of predetermined length, wherein a subset of consecutive elements in the vector are set to non-zero values corresponding to a time period of the indicated contrast phase, while remaining elements of the vector are set to zero. In a seventh example of the method, optionally including one or more or each of the first through sixth examples, the input priors further comprise a display field of view (DFOV) value and a reconstruction kernel type used to reconstruct the CT image. In a eighth example of the method, optionally including one or more or each of the first through seventh examples, the target energy level corresponds to an energy level within a continuous range between 40 keV and 140 keV. In a ninth example of the method, optionally including one or more or each of the first through eighth examples, the input energy spectrum comprises X-ray attenuation values across an energy range corresponding to the first energy level, and wherein the target energy spectrum comprises X-ray attenuation values across an energy range corresponding to the target energy level. In a tenth example of the method, optionally including one or more or each of the first through ninth examples, the neural network is trained using pairs of training images, wherein each pair comprises: a first training image acquired at the first energy level, a second training image acquired at the target energy level, wherein the first training image and the second training image are monochromatic images generated from dual-energy projection data, input priors comprising: an input energy spectrum corresponding to the first energy level, an input contrast phase timeline indicating a contrast phase of the first training image, a display field of view (DFOV) value used to reconstruct the first training image, and a reconstruction kernel type used to reconstruct the first training image, and target priors comprising: a target energy spectrum corresponding to the target energy level, a target contrast phase timeline matching the input contrast phase timeline, the DFOV value, and the reconstruction kernel type, wherein training comprises: processing the first training image and the input priors through the neural network to generate a transformed training image, determining a loss based on comparing the transformed training image to the second training image, and updating parameters of the neural network based on the loss to train the feature attention generator.

The disclosure also provides support for a system, comprising: a display device, one or more processors, and memory storing instructions executable by the one or more processors to: receive a computed tomography (CT) image acquired at a first energy level, obtain input priors including an input energy spectrum corresponding to the first energy level, obtain target priors including a target energy spectrum corresponding to a target energy level, process the CT image through a neural network to generate a transformed CT image at the target energy level, wherein the neural network produces at least one feature map, generate, using a feature attention generator, a feature conditioning vector based on the input priors and the target priors, adjust features of the at least one feature map using the feature conditioning vector to generate adjusted features, generate the transformed CT image using the adjusted features, and display the transformed CT image on the display device. In a first example of the system, the input priors further include an input contrast phase timeline indicating a contrast phase of the CT image and an input Display Field of View (DFOV), and wherein the target priors further include a target DFOV matching the input DFOV. In a second example of the system, optionally including the first example, the instructions to generate the feature conditioning vector include instructions to: process the input priors through a first multilayer perceptron (MLP) to generate first processed priors, process the target priors through a second MLP to generate second processed priors, concatenate the first processed priors and the second processed priors to form combined processed features, and process the combined processed features through a third MLP to generate the feature conditioning vector, wherein the feature conditioning vector has a dimension corresponding to a number of channels in the at least one feature map. In a third example of the system, optionally including one or both of the first and second examples, the instructions to adjust features of the at least one feature map using the feature conditioning vector include instructions to multiply each channel of the at least one feature map by a corresponding scalar value in the feature conditioning vector to generate weighted feature channels.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method, comprising:
receiving a computed tomography (CT) image acquired at a first energy level;
obtaining input priors including an input energy spectrum corresponding to the first energy level;
obtaining target priors including a target energy spectrum corresponding to a target energy level;
processing the CT image through a neural network to generate at least one feature map;
generating, using a feature attention generator, a feature conditioning vector based on the input priors and the target priors;
adjusting features of the at least one feature map using the feature conditioning vector to generate adjusted features; and
processing the adjusted features through the neural network to generate a transformed CT image at the target energy level.

2. The method of claim 1, wherein the input priors further include:
an input contrast phase timeline; and
an input Display Field of View (DFOV).

3. The method of claim 1, wherein generating the feature conditioning vector comprises:
processing the input priors through a first neural network to generate first processed priors;
processing the target priors through a second neural network to generate second processed priors;
concatenating the first processed priors and the second processed priors to form combined processed features; and
processing the combined processed features through a third neural network to generate the feature conditioning vector, wherein the feature conditioning vector has a dimension corresponding to a number of channels in the at least one feature map.

4. The method of claim 1, wherein adjusting features of the at least one feature map using the feature conditioning vector comprises modifying the at least one feature map through one or more operations based on numerical values of the feature conditioning vector.

5. The method of claim 4, wherein the one or more operations include multiplying each channel of the at least one feature map by a corresponding scalar value in the feature conditioning vector.

6. A method, comprising:
receiving a computed tomography (CT) image acquired at a first energy level;
obtaining input priors including an input energy spectrum corresponding to the first energy level;
obtaining target priors including a target energy spectrum corresponding to a target energy level;
processing the CT image through a neural network to generate a transformed CT image at the target energy level, wherein the neural network comprises:
an encoder pathway comprising a plurality of encoder levels, wherein each encoder level is configured to receive an input feature map and convolve the input feature map with one or more convolutional filters to generate an output encoder feature map; and
a decoder pathway comprising a plurality of decoder levels corresponding to the plurality of encoder levels, wherein each decoder level is configured to receive an input decoder feature map comprising an up-convolved feature map from a lower decoder level concatenated with the output encoder feature map from a corresponding encoder level;
generating at each decoder level, using a feature attention generator, a feature conditioning vector based on the input priors and the target priors;
adjusting features of the input decoder feature map at each decoder level using a corresponding feature conditioning vector to generate adjusted features;
convolving the adjusted features at each decoder level to generate an output decoder feature map; and
outputting the transformed CT image based on an output decoder feature map from a highest level of the decoder pathway.

7. The method of claim 6, wherein generating the feature conditioning vector at each decoder level comprises:
processing the input priors through a first MLP to generate processed input priors;
processing the target priors through a second MLP to generate processed target priors;
combining the processed input priors and processed target priors to generate combined features; and
processing the combined features through a third MLP to generate the feature conditioning vector.

8. The method of claim 7, wherein combining the processed input priors, and the processed target priors comprises concatenating the processed input priors, and processed target priors.

9. The method of claim 7, wherein adjusting features of the input decoder feature map comprises multiplying each channel of the input decoder feature map by a corresponding scalar value in the feature conditioning vector.

10. The method of claim 6, wherein the input priors further comprise a contrast phase timeline indicating a contrast phase of the CT image, and wherein the target priors further comprise a target contrast phase timeline.

11. The method of claim 10, wherein the contrast phase timeline comprises a one-dimensional curve indicating one or more of: a pulmonary artery phase, an aortic phase, a late arterial phase, a portal phase, a delayed phase, or a delayed equilibrium phase.

12. The method of claim 11, wherein the contrast phase timeline is encoded as a vector of predetermined length, wherein a subset of consecutive elements in the vector are set to non-zero values corresponding to a time period of the indicated contrast phase, while remaining elements of the vector are set to zero.

13. The method of claim 6, wherein the input priors further comprise a display field of view (DFOV) value and a reconstruction kernel type used to reconstruct the CT image.

14. The method of claim 6, wherein the target energy level corresponds to an energy level within a continuous range between 40 keV and 140 keV.

15. The method of claim 6, wherein the input energy spectrum comprises X-ray intensity values across an energy range corresponding to the first energy level, and wherein the target energy spectrum comprises X-ray intensity values across an energy range corresponding to the target energy level.

16. The method of claim 6, wherein the neural network is trained using pairs of training images, wherein each pair comprises:
a first training image acquired at the first energy level;
a second training image acquired at the target energy level, wherein the first training image and the second training image are monochromatic images generated from dual-energy projection data;
input priors comprising:
an input energy spectrum corresponding to the first energy level;
an input contrast phase timeline indicating a contrast phase of the first training image;
a display field of view (DFOV) value used to reconstruct the first training image; and
a reconstruction kernel type used to reconstruct the first training image; and
target priors comprising:
a target energy spectrum corresponding to the target energy level;
a target contrast phase timeline matching the input contrast phase timeline;
the DFOV value; and
the reconstruction kernel type;
wherein training comprises:
processing the first training image and the input priors through the neural network to generate a transformed training image;
determining a loss based on comparing the transformed training image to the second training image; and
updating parameters of the neural network based on the loss to train the feature attention generator.

17. A system, comprising:
a display device;
one or more processors; and
memory storing instructions executable by the one or more processors to:
receive a computed tomography (CT) image acquired at a first energy level;
obtain input priors including an input energy spectrum corresponding to the first energy level;
obtain target priors including a target energy spectrum corresponding to a target energy level;
process the CT image through a neural network to generate a transformed CT image at the target energy level, wherein the neural network produces at least one feature map;
generate, using a feature attention generator, a feature conditioning vector based on the input priors and the target priors;
adjust features of the at least one feature map using the feature conditioning vector to generate adjusted features;
generate the transformed CT image using the adjusted features; and
display the transformed CT image on the display device.

18. The system of claim 17, wherein the input priors further include an input contrast phase timeline indicating a contrast phase of the CT image and an input Display Field of View (DFOV), and wherein the target priors further include a target DFOV matching the input DFOV.

19. The system of claim 17, wherein the instructions to generate the feature conditioning vector include instructions to:
process the input priors through a first multilayer perceptron (MLP) to generate first processed priors;
process the target priors through a second MLP to generate second processed priors;
concatenate the first processed priors and the second processed priors to form combined processed features; and
process the combined processed features through a third MLP to generate the feature conditioning vector, wherein the feature conditioning vector has a dimension corresponding to a number of channels in the at least one feature map.

20. The system of claim 17, wherein the instructions to adjust features of the at least one feature map using the feature conditioning vector include instructions to multiply each channel of the at least one feature map by a corresponding scalar value in the feature conditioning vector to generate weighted feature channels.
